(19) United States
(12) Patent Application Publication
(10) Pub. No.: US 2025/0123263 A1
(43) Pub. Date: Apr. 17, 2025

(54) Coupled Nanopores For Molecular Analysis

(71) Applicants: The Trustees of the University of Pennsylvvania, Philadelphia, PA (US); The Children's Hospital of Philadelphia, Philadelphia, PA (US)

(72) Inventors: Marija Drndic, Philadelphia, PA (US); Chih-Yuan Lin, Philadelphia, PA (US); Yung-Chien Chou, Philadelphia, PA (US); Dimitrios S. Monos, Merion Station, PA (US)

(21) Appl. No.: 18/913,088

(22) Filed: Oct. 11, 2024

Related U.S. Application Data
(60) Provisional application No. 63/589,407, filed on Oct. 11, 2023.

(51) Int. Cl. G01N 33/487; B82Y 15/00

(52) U.S. Cl. CPC G01N 33/48721; B82Y 15/00

(57) ABSTRACT

A molecular analysis component, comprising: a first substrate having a first nanopore extending therethrough and the first nanopore having a diameter; and a second substrate having a second nanopore extending therethrough and the second nanopore having a diameter, the first and second nanopores both extending in a direction, and the first and second nanopores are separated by a distance of from about 0.5 nm to about 500 nm as measured along the direction, the distance optionally being of from about 5 nm to about 250 nm. A method, comprising: translocating a molecule through (i) a first nanopore extending through a first substrate and (ii) a second nanopore extending through a second substrate, the first and second nanopores both extending along a direction, the first and second nanopores being separated by a distance as measured along the direction; and collecting at least one signal related to the translocation of the molecule through at least one of the first nanopore and the second nanopore, the molecule optionally comprising a polynucleotide.

### CROSS-REFERENCE TO RELATED APPLICATIONS

This application claims the benefit of and priority to U.S. Provisional Application No. 63/589,407, filed Oct. 11, 2023, the entirety of which application is incorporated by reference herein for any and all purposes.

### GOVERNMENT RIGHTS

This invention was made with government support under HG010536, and HG012395 by the National Institutes of Health, and 1905045 and 2002477 awarded by the National Science Foundation. The government has certain rights in the invention.

### TECHNICAL FIELD

The present disclosure relates to the field of nanopore devices and to the field of macromolecular analysis.

### BACKGROUND

The designs of certain existing nanopore devices used to obtain molecular information (e.g., ssDNA sequencing) do not always allow for elongation and/or straightening of the molecules under study. This lack of elongation can, in some cases, result in collection of incomplete information from the molecules under study. Accordingly, there is a need in the art for alternative nanopore device designs, the value of which designs would be enhanced by the designs' ability to elongate the molecules under study.

### SUMMARY

In meeting the described need in the art, the present disclosure provides a molecular analysis component, comprising: a first substrate having a first nanopore extending therethrough and the first nanopore having a diameter; and a second substrate having a second nanopore extending therethrough and the second nanopore having a diameter, the first and second nanopores both extending in a direction, and the first and second nanopores are separated by a distance of from about 0.5 nm to about 500 nm as measured along the direction, the distance optionally being of from about 5 to about 250 nm.

Also provided is method, comprising translocating a molecule through the molecular analysis component of the present disclosure, e.g., according to any one of Aspects 1 to 13 presented herein.

Further provided is a method, comprising: translocating a molecule through (i) a first nanopore extending through a first substrate and (ii) a second nanopore extending through a second substrate, the first and second nanopores both extending along a direction, the first and second nanopores being separated by a distance as measured along the direction; and collecting at least one signal related to the translocation of the molecule through at least one of the first nanopore and the second nanopore, the molecule optionally comprising a polynucleotide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1D. Coupled bilayer nanopores for guiding, tracking and sizing of single molecules. (FIG. 1A) Fabrication schematic including electron beam lithography (EBL), etching, 2D materials transfer, and aberration-corrected transmission electron microscopy (AC-TEM) sculpting. SiN nanopores constitute a guiding and reusable (GURU) platform. Pore thicknesses are t_{SiN} and t_{2D}, diameters d_{SiN} and d_{2D}, and the total thickness is t_{GURU}. Device configurations are denoted as [N,M] for N SiN pores and M 2D pores. In this work, M=1. M=0, when 2D layer is absent. (FIG. 1B) Electric field profiles for [1,1]. d_{2D}=6.5 nm, d_{SiN}=15 nm, t_{2D}=1 nm, t_{SiN}=7 nm. L=2, 20 nm (coupling) and 200 nm (decoupling regime). Conductance ratio of G(L)/G(L=200 nm) and ΔG/ΔL for [1,1] vs. L; ΔG/ΔL˜1 nS/nm for L˜10 nm. (FIG. 1C) Conductance regimes and [N,1] configurations: G_{GURU}<<G_{2D}, G_{GURU}˜G_{2D}, and G_{GURU}>>G_{2D}. (FIG. 1D) G_{GURU}/G_{2D} vs. d_{SiN}/d_{2D} and t_{SiN}/t_{2D} for [1,1], and G_{GURU}/G_{2D} vs. d_{SiN}/d_{2D} and N for [N,1]. L=20 nm. Fixed parameters are indicated. G_{GURU}=G_{2D} is outlined with red-dashed lines.

FIGS. 2A-2E. [N,1] configurations: TEM images, modeling, and measurements. (FIG. 2A) Bright-field (BF) TEM images of GURU layers: L=20±1 nm for [1,0] to [3,0] and [9,0], L=100±1 nm for [4,0]. EBL/etching is used for [9,0], TEM drilling for [1,0] to [3,0], and a combination of both for [4,0]. Yellow dashed square indicates where TEM drilling is used. Image of [9,1] after reusing it for four times (orange rectangle). (FIG. 2B) Dark-field (DF) AC-STEM images (xz-plane) of Chip J, [6,1], with a monolayer (2D) MoS₂ nanopore, d_{2D}(TEM)=2.4±0.5 nm (red oval). Images are focused on the bottom of the SiN layer. Insets are fast Fourier transforms (FFTs) of the hexagonal 2D lattice before and after pore formation. The 2D pore is drilled below Pore 2 (light-green square). (FIG. 2C) Simulated electric field profiles of [6,1] in xy-planes containing Pores 1-3 and 4-6, respectively. Geometric parameters are indicated, and L=20 nm. (FIG. 2D) Data from Chip J with 90-nt ssDNA. Current-time trace at 300 mV and 1 M KCl, recorded at 1 MHz bandwidth and Bessel filtered at 100 kHz. G=13.7 nS. L=20±1 nm, t_{GURU}=40±3 nm, and d_{SiN}(TEM)=31.0±0.9 nm. For [6,0], G_{GURU}=2.2 μS>>G_{2D}. Iᵣₘₛ at 0 V is shown for [6,1] and [6,0]. (FIG. 2E) Data from 90-nt ssDNA with Chip K, [4,1]. Current-time trace showing current enhancement (“up”) events at 500 mV and 1 M KCl recorded at 1 MHz bandwidth, and Bessel filtered at 100 kHz. G=11.0 nS, L=20±1 nm, t_{GURU}=40±3 nm, and d_{SiN}=24.6±2.0 nm. See Methods for diameter and error analysis.

FIGS. 3A-3D. Simulations of two decoupled pores, and coupled configurations [1,1] and [6,1]. (FIG. 3A) Simulated ionic currents vs. double-stranded DNA (dsDNA) position along y-direction assuming straight trajectories. (Top) Y_{DNA} is the coordinate of the dsDNA front. dsDNA is simulated as a charged rod entering from the GURU side (Y_{DNA}=0). L_{DNA} is the DNA length. (Middle) Decoupling regime of distant pores for [1,1] as in FIG. 1b, t_{GURU}=107 nm, showing two separated U-events. (Bottom) Coupling regime for [6,1] as in FIGS. 2A-2E, showing one U-event from the 2D pore (t_{GURU}=27 nm). Pore diameters (d), thicknesses (t), and y-positions are indicated, V=200 mV. (FIG. 3B) Evolution from U to W (L_{DNA}<t_{GURU}) and from U to T events (L_{DNA}>t_{GURU}) for [1,1] for d_{2D}=3-14 nm. Pore dimensions and pore y-positions are indicated, V=200 mV. Maximum intra-event magnitudes are normalized to 1. The solid color in the schematic marks what is varied. (FIG. 3C) Evolution from W to T events for L_{DNA}=10-50 nm (ΔL_{DNA}=10 nm), and L_{DNA}=100 nm for [1,1], L=20 nm. Pore geometries and y-positions are indicated. V=300 mV (Left) and 200 mV (Right). Maximum inter-event magnitude is normalized to 1 as L_{DNA}=100 nm (>t_{GURU}). (FIG. 3D) Simulated event blockade illustrates an example of symmetric T₁ and T₃ sublevels; configurations are shown as indicated. (Right) Spatial profiles (i to ix) of the total concentration, Σcᵢ, of potassium and chloride ions (see Methods) for the corresponding Y_{DNA} as DNA molecule translocates. The maximum event magnitude is normalized to 1.

FIGS. 4A-4L. Ionic experiments with [1,1] configuration. (FIG. 4A) Bright field (BF) and dark field (DF) TEM/AC-STEM images of [1,1], G_{2D}˜G_{GURU}. The as-fabricated TEM-measured pore diameters for Chip L were d_{2D}(TEM)=2.8±0.7 nm, d_{SiN}(TEM)=14.0±0.9 nm, t_{GURU}=40±3 nm, L=20±1 nm. Ionic current vs. time for open-pore current at 0 V and after adding 1500-bp dsDNA at 50, 100, 150, and 200 mV in 1 M KCl. Inset: conventional shapes for unfolded and partially folded DNA translocation events are acquired from a single comparable, small and thin, nanopore. (FIG. 4B) T- and W-events. At V=200 mV, T-events for 1500-bp dsDNA and W-events for ˜100-bp dsDNA; W-events for 200-nt ssDNA. 1 M KCl, 1 MHz bandwidth, Bessel filtered at 100 kHz. (FIG. 4C, FIG. 4D) Schematics illustrating DNA translocations and boxplots of event depths, T₁ to T₃ and W₁ to W₃ for data in (FIG. 4B). (FIG. 4E, FIG. 4F) Schematics for Chips M and N (FIG. 4G, FIG. 4H) and their corresponding simulated electric field profiles. (FIG. 4I, FIG. 4J) Boxplots for sublevel event dwell time T₀₂ and T₂₄ at various voltages for Chips M and N. (FIG. 4K, FIG. 4L1) Sample T-events from Chips M and N, at V=800 mV for 200-bp dsDNA, 3 M KCl, 10 MHz bandwidth, Bessel filtered at 1 MHz.

FIGS. 5A-5B. 3D model using finite element analysis to simulate the open pore currents and current blockades when DNA molecules translocate in the 2D-GURU device (not to scale). Y_{DNA} is the coordinate of the DNA front. (FIG. 5A) [N, 1]=[1,1]; (FIG. 5B) [N, 1]=[6, 1]. N denotes the number of SiN pores in the bottom layer.

FIGS. 6A-6F. Nanopore size control throughout the STEM drilling process. Series of ADF-STEM (annular dark field scanning transmission electron microscopy) images of suspended monolayer MoS₂ during the nanopore drilling process on two different devices. Some accumulation of atoms on the pore edges is visible and this can contribute to an increased effective thickness of the pore in some cases. (FIG. 6A) MoS₂ lattice before drilling, (FIG. 6B) after a few seconds of drilling, a sub-nanometer pore is created (size˜0.85 nm×0.85 nm), (FIG. 6C) same nanopore after a few additional seconds of drilling, yielding a final nanopore with a size adapted to ssDNA translocation measurements (˜1.7 nm×1.3 nm). This pore is a part of a [6,1] device configuration. (FIG. 6D-FIG. 6F) Images of the same process on another GURU device, aiming for a nanopore size adapted to translocation measurements of dsDNA. Pore sizes are ˜1.29 nm×1.47 nm, 1.81 nm×1.89 nm, 2.17 nm×3.15 nm for D, E and F, respectively. These sizes are extracted from two intensity profiles taken at the widest points of the pore vertically and horizontally, respectively. All scale bars are 2 nm.

FIGS. 7A-7F. Additional examples of MoS₂ nanopore drilling for three other GURU devices with varying geometries. (FIG. 7A, FIG. 7B) [2,1], (FIG. 7C, FIG. 7D [6,1], (E, F) [9,1]. ADF-STEM (annular dark field scanning transmission electron microscope) images of (FIG. 7A) RIE-thinned region of the SiN membrane containing two TEM-drilled nanopores (inset: closeup of the bottom nanopore covered by monolayer MoS₂ prior to drilling; (FIG. 7B) 2D nanopore; (FIG. 7C) pre-patterned RIE-thinned square in the SiN membrane containing six EBL nanopores, (FIG. 7D) closeup of pore highlighted by the blue dashed square in (FIG. 7C), showing the suspended monolayer MoS₂ after STEM drilling (inset: high resolution image of the 2D nanopore), (FIG. 7E) pre-patterned RIE-thinned square in the SiN membrane containing nine EBL nanopores, (FIG. 7F) closeup of pore highlighted by the blue dashed square in (FIG. 7E) showing the suspended monolayer MoS₂ after STEM drilling (inset: high resolution image of the 2D nanopore). FIG. 7E and FIG. 7F are images of the reused device shown in FIG. 2A in the main text, the STEM drilling step shown here corresponds to the fourth MoS₂ transfer process, and third STEM drilling that this device underwent. Scale bars are 100 nm for FIG. 7A, FIG. 7B and FIG. 7C, 5 nm for FIG. 7D, FIG. 7E and FIG. 7F, and 2 nm in all inset images.

FIGS. 8A-8C. (FIG. 8A) Additional images of [6,1] from FIG. 2b; (FIG. 8B) Additional images of [4,1] from FIG. 2e; (FIG. 8C) Additional images of [1,1] from FIG. 4, before and after 2D pore formation.

FIGS. 9A-9B. Additional images of GURU devices with [1,1] configuration for FIG. 4E, (FIG. 9A), and FIG. 4I, (FIG. 9B). (FIG. 9A) d_{2D}(TEM)˜2 nm, d_{SiN}(TEM)=12 nm. (B) d_{2D}(TEM)˜2 nm, d_{SiN}(TEM)=9 nm.

FIGS. 10A-10B. (FIG. 10A) TEM images showing two patterned windows on top of which 2D material is deposited. The 2D pores can be selectively drilled in multiple desired locations. (FIG. 10B) Series of TEM images (top) and optical images (bottom) after the same device was reused. On this device we deposited ALD (atomic layer deposition) HfO₂ (˜3 nm) after the first round of measurement, which can be seen as the white circles around the pore edges. The position of two patterned windows is highlighted in dotted red squares. Devices presented in the main text do not contain the HfO₂ layer and this device in FIG. 10B was used for wetting and ionic measurement tests.

FIGS. 11A-11B. Color maps of open pore conductance G₀ in 1M KCl as a function of pore diameters, d, and pore thicknesses, t, (FIG. 11A) for the 2D pore, and (FIG. 11B) for the SiN pore.

FIGS. 12A-12B. Conductance blockade, ΔG, from unfolded dsDNA in 1 M KCl through (FIG. 12A) a single 2D pore, and (FIG. 12B) a single SiN pore for a range of pore thicknesses and diameters.

FIGS. 13A-13D. Examples of variation in T₁ and T₃ with different voltages when a 50-nm dsDNA translocates through a 2D-GURU device. (FIG. 13A) d_{2D}=3 nm, d_{SiN}=5 nm, t_{2D}=1 nm, t_{SiN=}5 nm; (FIG. 13B) d_{2D}=8 nm, d_{SiN}=15 nm, t_{2D}=1 nm, t_{SiN}=7 nm. The surface charge densities assumed in the simulation are σ_{SiN}=σ_{2D}=−0.02 C/m². These are not well known in each experiment and can probably vary due to pore conditions and preparation. Applied voltages are 0.1V (Red), 0.2V (black), and 0.3V (blue). (FIG. 13C, FIG. 13D) Corresponding concentration profiles for various Y_{DNA} at V=0.1 V and 0.2 V in FIG. 13A. Note that Σcᵢ=c_{K+}+C_{cl-} represents the sum of the concentration of potassium and chloride ions. The total concentration eventually reaches its bulk value of 2M (i.e., 1M K⁺ ions+1M Cl⁻ ions) for the points far away from the pore (teal/green-colored area below and above the pores in FIG. 13C and FIG. 13D).

FIG. 14. Normalized event depth vs. the length of DNA, L_{DNA}, at given positions of Y_{DNA}=0, 10, 20, 40 nm for the GURU device demonstrated in FIG. 3C (left). The same normalization method is applied here. Taking the cases where Y_{DNA}>t_{GURU} as a universal normalized scale across various Y_{DNA}, the baseline current and the maximum event depth (T₂) are normalized to 0 and 1, respectively, to focus on the comparison of event signal shapes.

FIG. 15. Example of long T-shaped events from the [1,1] configuration, discussed in FIGS. 4A-4C. Measured current vs. time for ˜1500-bp dsDNA in at 50 mV. These event durations are similar to the Zimm relaxation time¹³. The yellow bands highlight the T₁, T₂, T₃ levels.

FIG. 16. Gel electrophoresis result from (B-I) samples containing varying concentrations of 1500-bp and 100-bp dsDNA, and (J) the translocation sample used in the ionic measurements.

FIGS. 17A-17B. Simulated current change for 50-nm DNA passing through single nanopores. (FIG. 17A) 3 nm in diameter and 1 nm in thickness for single 2D pore; (FIG. 17B) 15 nm in diameter and 20 nm in thickness for single SiN pore.

FIGS. 18A-18B. (FIG. 18A) Schematics of cylindrical and hourglass pore geometrical model used in the simulation. (FIG. 18B) Comparison of current change between two different geometric models. Grey-dashed curve: an equivalent cylindrical pore of effective thickness (i.e., 7 nm); blue curve: an hourglass-shaped pore of 20 nm thickness in total. The applied voltage is 0.2 V and L_{DNA}=50 nm.

FIG. 19. Normalized current change in a [1,1] configuration for 50-nm-long polymer with a higher surface charge density of −0.1 C/m². The applied voltage is 0.2 V. The geometric parameters are: [d_{2D}, t_{2D}]=[6.5 nm, 1 nm], [d_{SiN}, t_{SiN}]=[15 nm, 20 nm], and L=20 nm. The Y axis and details are defined in the modeling as in FIGS. 5A-5B. We note that ΔI is the maximum value of the blockade and is normalized to 1. The negative sign indicates the current enhancement. The current blockade occurs in the SiN pore and the current enhancement occurs when the DNA enters the 2D pore.

FIG. 20. Feasibility and schematic illustration of multiple layer GURU devices with one more pore within each respective layer.

FIG. 21. A schematic of the tubular furnace, substrate, and sulfur source is shown.

FIGS. 22A-22D. Example of pore extractions for area measurement via commercial software package Image J. Extracted area is outlined in white dotted line on the right. The calculated pore diameters are (FIG. 22A) d_{2D}(TEM)=1.6±0.2 nm, (FIG. 22B) d_{SiN}(TEM)=24.6±2.0 nm for the device shown in FIG. 2E, and (FIG. 22C) d_{2D}(TEM)=2.8±0.7 nm, (FIG. 22D) d_{SiN}(TEM)=14.0±0.9 nm for the device shown in FIG. 4A.

FIGS. 23A-23B. Noise performance of the GURU devices. (FIG. 23A) Input-referred current noise PSD with various configurations N=[1,1], [6,0], and [6,1]. (FIG. 23B) Concatenated time trace of a 200 ms long open-pore measurements. The same trace is filtered by a digital four-pole Bessel filter to cutoff frequency, from 1 MHz to 100 KHz. The respective IRMS values for N=[1,1], [6,0], and [6,1] are 57.1 pAᵣₘₛ, 85.4 pAᵣₘₛ and 40.7 pAᵣₘₛ.

FIGS. 24A-24B. Flow cell schematic and assembly for (FIG. 24A) conventional PDMS flow cell, and (FIG. 24B) customized PMMA flow cell. Both are used to accommodate the 5 by 5 mm² GURU chips.

FIG. 25. Concatenated current trace over time from FIG. 4A at 200 mV and FIG. 4E at 800 mV.

FIGS. 26A-26B. (FIG. 26A) Event depth for T- and W-events (sublevels T₁-₃ and W₁-₃) vs. voltage for data in FIG. 4. (FIG. 26B) FWHM and Mean of the segmented dwell time distributions as fitted in FIG. 4F.

FIG. 27. Flow chart outlining the analysis procedure for event detection and characterization. Here I₀ is the calibrated baseline currents. fₛ and f_{c} are the recording and cut-off frequency, respectively.

FIG. 28. Exemplary component according to present disclosure.

FIG. 29. Exemplary component according to present disclosure.

FIG. 30. Exemplary component according to present disclosure.

FIG. 31. Exemplary component according to present disclosure.

FIG. 32. Exemplary component according to present disclosure.

FIGS. 33A-33D. (FIG. 33A) Event depth for T- and W-events (sublevels T₁-₃ and W₁-₃) vs. voltage for exemplary data. data in FIG. 4D. The box ranges from the first to the third interquartile, and the median is indicated by a horizontal line within the box, with whiskers extending to the minimum and maximum values of the dataset. For T-events, n=89, 132, and 168 for 50, 100, and 150 mV, respectively. For W-events, n=45, 60, and 84 for 50, 100, and 150 mV, respectively. (FIG. 33B, FIG. 33C) FWHM and Mean of the segmented dwell time distributions. (FIG. 33D) Full and segmented mean event durations at 100, 150, and 200 mV within T- and W-events for dsDNA data in FIG. 4D. Indices 0-4 are used to indicate various sublevels and translocation stages. The coloured bars and error bars show the mean±s.d.

FIGS. 34A-34F. (FIG. 34A) Optical image of device. Red region denotes the suspended SiNx membrane. Thinned SiNx denotes the local-thinned trench, which is covered by the hBN flake (pink outline). (FIG. 34B) AFM (Atomic force microscopy) image displays the hBN flake is approximately 11 nm thick. Blue box represents the scanned region. (FIG. 34C) STEM (scanning transmission electron microscope) image illustrates the pore array in the bottom layer. It contains 20 pores separated by 100 nm in a 400nm×400 nm square (black region). (FIG. 34D) STEM (scanning transmission electron microscope) image of a 3 nm diameter nanopore drilled in the top hBN layer. Drilling was performed on a Cs-corrected JEOL NEOARM operating at 200 kV. (FIG. 34E) A 5 second current trace recorded using 10 MHz bandwidth amplifier with low-pass filter at a cutoff frequency of 100 kHz. The applied voltage is 500 mV. The electrolyte solution is 1M KCl and pH 8. The concentration of DNA is 2.2 nM. (FIG. 34F) Translocation event examples for 15 kbp dsDNA that extracted from the current trace. (Top) two-step events; (bottom) single-file event.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Rapid sensing of molecules is increasingly important in many studies and applications. Beyond atomically thin (2D) nanopores, here, we conceptualize, simulate, and experimentally demonstrate coupled, guiding, and reusable bilayer nanopore platforms (GURU), enabling advanced ultrafast detection of unmodified molecules. The bottom layer collimates and decelerates the molecules under detection, and the top 2D pore enables sensing. We vary the number of pores in the bottom layer from nine to one while fixing one 2D pore in the top layer. When the number of pores in the bottom layer is also reduced to one, sensing is enabled by both layers, and distinct T- and W-shaped signals indicate the position of molecules and discriminate their lengths. This is uniquely enabled by microsecond resolution capabilities and precise nanofabrication. These coupled nanopores represent configurable multifunctional systems with inter- and intra-layer structures for electromechanical control, filtration, and prolonged dwell time in a desired sensing zone.

Detecting single molecules with electrical and optical approaches has advanced understanding of fundamental processes and the correlation of structure and properties at the single-particle level¹. The idea of counting particles through holes grew from the 1950s into resistive-pulse sensing, where driven molecules produce current blockades. This broad concept has allowed enzyme-assisted DNA sequencing², biomarker detection, and usage in filtration and desalination³⁻⁷. Labeling molecules with markers such as nanoparticles and attaching them to carriers, such as dsDNA, have been some of the proxy approaches to increase sensitivity⁸ while reducing the need for small pores. However, these approaches require sample modification, which often alter interactions and dynamics. Alternatively, or in addition, designing internal pore layers and scaling pores down to atomic scale while simultaneously improving signal-to-noise ratio and time resolution would enable direct and ultrafast reading of unmodified molecules and their dynamics^{9,10} at unprecedented resolution. At typical translocation rates of 0.1-1 μs/bp⁹, DNA travels a 10 nm distance in ˜3-30 μs, which is now within the experimental resolution reach. Integrating guiding structures at this scale into pores can facilitate regional velocity control on translocating molecules.

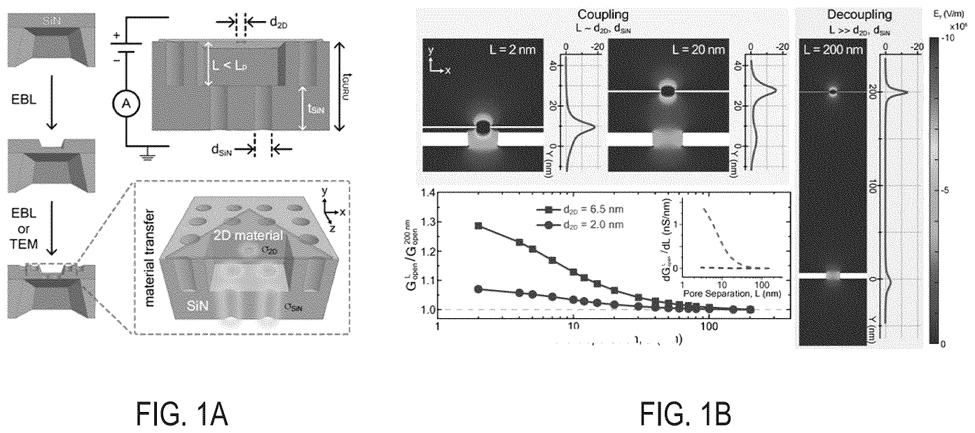

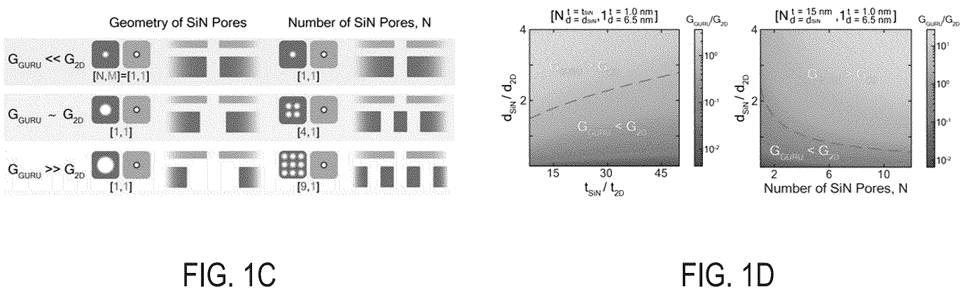

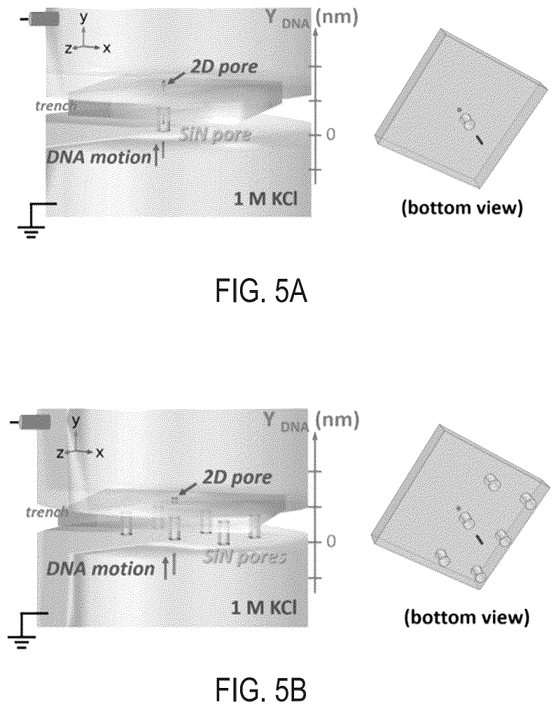

We simulate and demonstrate a general bilayer coupled nanopore concept for ultrafast guiding, tracking, and detection of single unmodified molecules, placing pores in two layers with configurable structures at nanometer precision (FIG. 1A). One or more pores can be placed within each layer, and pore conductance is tailored via their geometry and charge. Different sensor regimes are achieved by varying the number of pores in the bottom layer from several (nine in this work) to one, while restricting the number in the top layer to one 2D pore. When inter-layer separation, L, is comparable to or smaller than the persistence length of polymers, Lₚ (e.g., dsDNA), the polymer behaves locally like a rigid rod. The flexible design of the coupled nanopores where L<Lₚ provides capabilities to constrain polymers before entering the final sensing layer. As unique components for atom-scale engineering, 2D materials are optimal in spatial resolutions¹¹ and stackable modularity¹² for the bilayer coupled nanopore design. Here, we position a 2D monolayer above a novel guiding and reusable (GURU) silicon nitride (SiN) layer that can be as thin as 1 nm^{13,14}. [N, M=1] in FIG. 1A denotes N pores in the bottom GURU layer and one 2D pore in the top layer with surface charges σ_{SiN} and σ_{2D} (FIG. 5). Geometric parameters (FIG. 1A) are experimentally controlled with nanometer precision using lithography, etching, sculpting¹⁵⁻¹⁷, and surface charges via material choice.

### Coupling of Electric Fields

When two pores are placed in close vicinity (L˜d_{2D}, d_{SiN}), their electric fields overlap and couple (FIG. 1B, d_{2D}=6.5 nm, d_{SiN}=15 nm). In our work, L and d are >100 and 10 times smaller than those shown previously^{18,19}, demonstrating nontrivial coupling effects for the first time. Conductance G increases as L decreases to ˜d_{2D}, d_{SiN}, and G>(G_{GURU}G_{2D})/(G_{GURU}+G_{2D}) (coupled regime), marking a departure from decoupled pores (L>>d_{2D}, d_{SiN}). The slope ΔG/ΔL˜1 nS/nm at L˜10 nm (d_{2D}/d_{SiN}=6.5 nm/15.0 nm=0.43) shows how G is sensitive to nm-size changes in L, presenting a strategy to exploit pore coupling to probe sub-nm membrane vibrations electrically. FIG. 1C shows characteristic regimes and configurations: G_{GURU}<<G_{2D}, G_{GURU}˜G_{2D}, and G_{GURU}>>G_{2D}, (e.g., with fixed d_{2D}=6.5 nm, t_{2D}=1 nm (FIG. 1D), the regime G_{2D}˜G_{GURU} can be reached by varying N and/or d_{SiN}).

### Guiding and Reusable [N, 1] Nanopore Systems

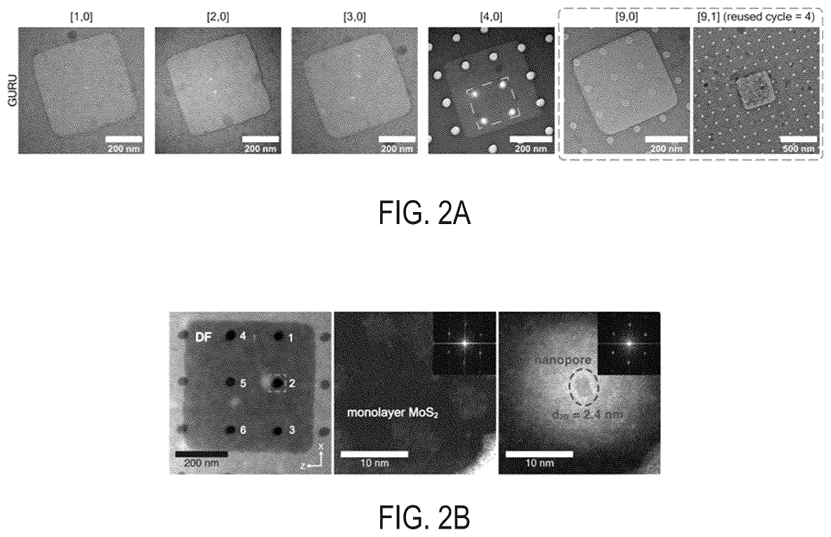

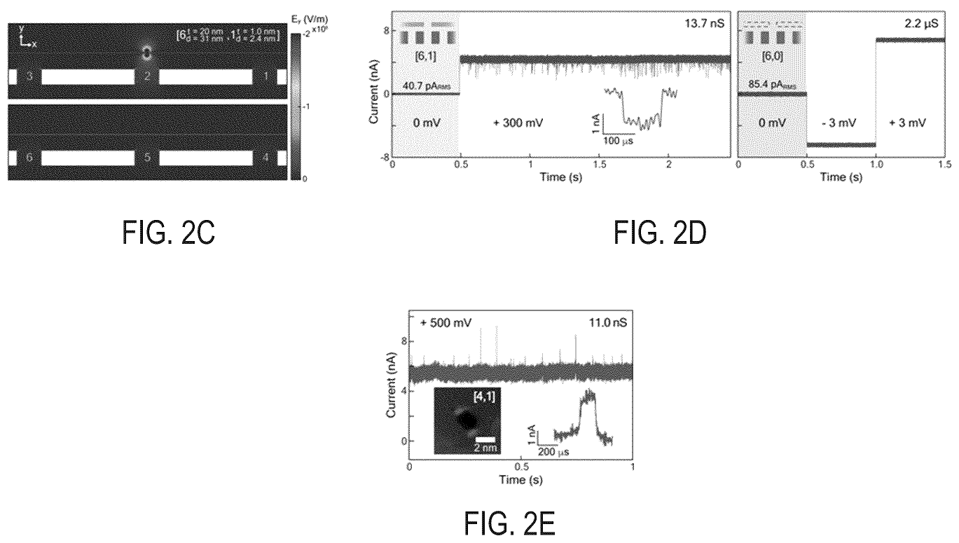

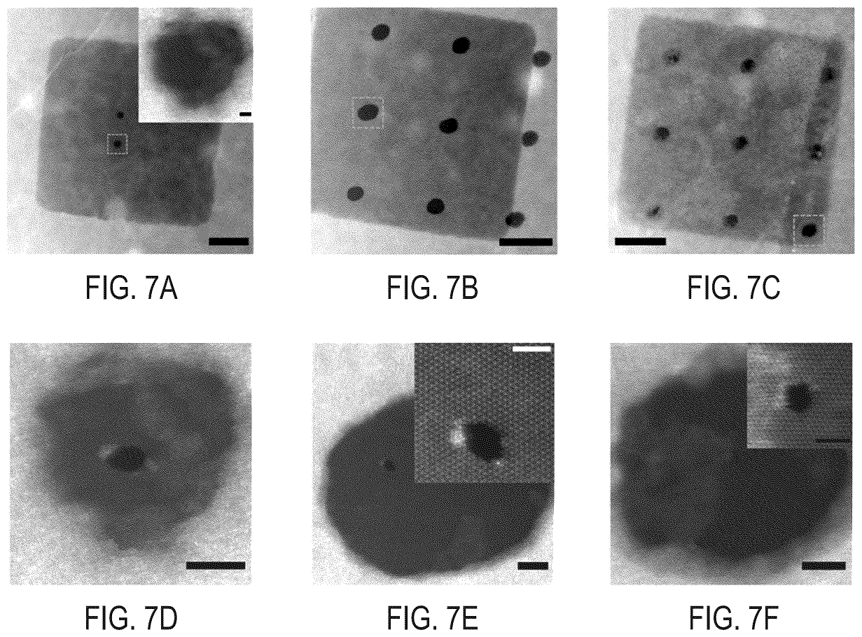

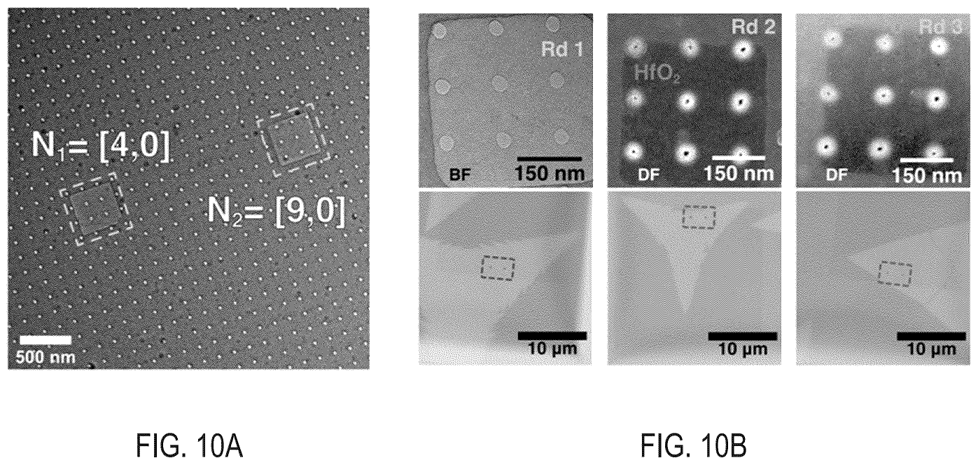

GURU layers are configurable. FIG. 2A shows TEM images of SiN trenches of varying t_{GURU}, d_{SiN} and t_{SiN}, made with TEM drilling (Chip A [1,0], Chip B [2,0], Chip C [3,0]), electron beam lithography (EBL) and reactive ion etching (RIE) (Chip D [9,0]), or their combination (Chip E [4,0]). The precision of RIE anisotropic etching allows only selected areas to have pores (Chip D [9,0] and Chip J [6,0], FIG. 2B). Assisted with TEM ([4,0]), we bypass EBL limitations to fabricate smaller nanopores. GURU platforms are reusable, as exemplified by [9,1], where Chip D was reused four times (four cycles of 2D material transfer, eight cycles of piranha surface treatment). The gradual sub-nm drilling process is shown in FIGS. 7-9. Successful storage, wetting, and low-noise stable performance were developed (see Methods). Wafer-scale integration is viable with multiple trench patterning (FIG. 10A) and proper nanofluidic design. Some samples used for ionic testing were successfully coated with a thin layer of HfO₂ for additional robustness and to causally tailor pore sizes (FIG. 10B).

The geometry with multiple pores in the bottom layer, N>1, is optimized for molecule guiding, while sensing is enabled by the top 2D pore. DNA pre-confinement was previously shown using a nanoporous filtering membrane placed at an order of magnitude larger distance (200 nm) from 6.7 to 8.0-nm-diameter SiN pore, reporting a suppressed frequency of folded DNA translocations²⁰. FIG. 2B shows AC-STEM images of Chip J, [6, 1], with a 2D MoS₂ nanopore (d_{2D}(TEM)=2.4±0.5 nm). Here, L=20±1 nm, t_{GURU}=40±3 nm, and d_{SiN}(TEM)=31.0±0.9 nm. Diameter errors are included from TEM images only and reflect the as-fabricated pores. However, we note the limitation of TEM-diameters as pores could, in principle, change over time and measurements. The GURU layer resistance is negligible (G_{2D}<<G_{GURU}). The open-pore conductance G˜G_{2D}=13.7 nS in 1 M KCl, in agreement with the calculated value assuming t_{2D}=2.2±1.2 nm, where the error in t_{2D} is calculated from the error in TEM-estimated diameter (see Methods). Small d_{2D} forces dsDNA to pass unfolded. The coupled electric field profiles (FIG. 2C) are mapped accordingly. FIGS. 2D and 2E demonstrate the detection of 90-nucleotide (nt) ssDNA. A 3-second-long current-time trace, measured at 300 mV in 1 M KCl, is displayed (FIG. 2D) from a 70-minute experiment. The 2D flake is annealed to the SiN layer, where the suspended area is minimized to ensure robustness and a stable current is observed. Chemically removing the 2D material results in a noticeable increase in G_{GURU}([6,0])=2.2 μS, matching the calculated value from the sum of six SiN pores since G_{GURU} increases with N and d_{SiN}¹⁰. Another dataset, Chip K, with [4,1] is shown in FIG. 2E. The 2D pore diameter d_{2D}(TEM)=1.6±0.2 nm and the open-pore conductance is only G=11 nS and corresponds to a calculated diameter d_{2D}=1.6 nm, assuming t_{2D}=1 nm. We detect current enhancement events; to our knowledge, this is the smallest diameter 2D pore measured for translocations. “Up” events for dsDNA were previously reported in a SiN pore²¹ and glass pipette pores²².

### Principle of Advanced Tracking and Sizing of Molecules with [1,1]

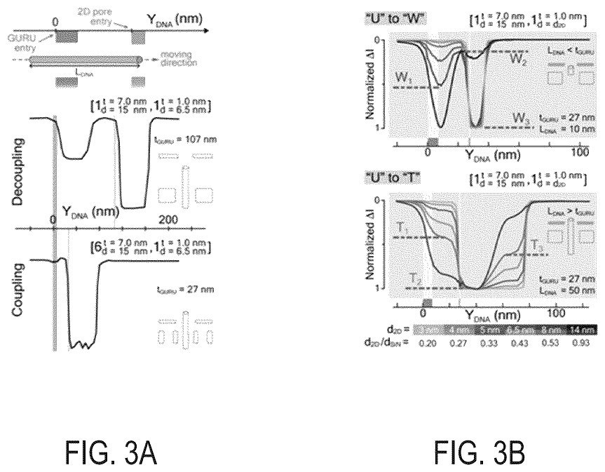

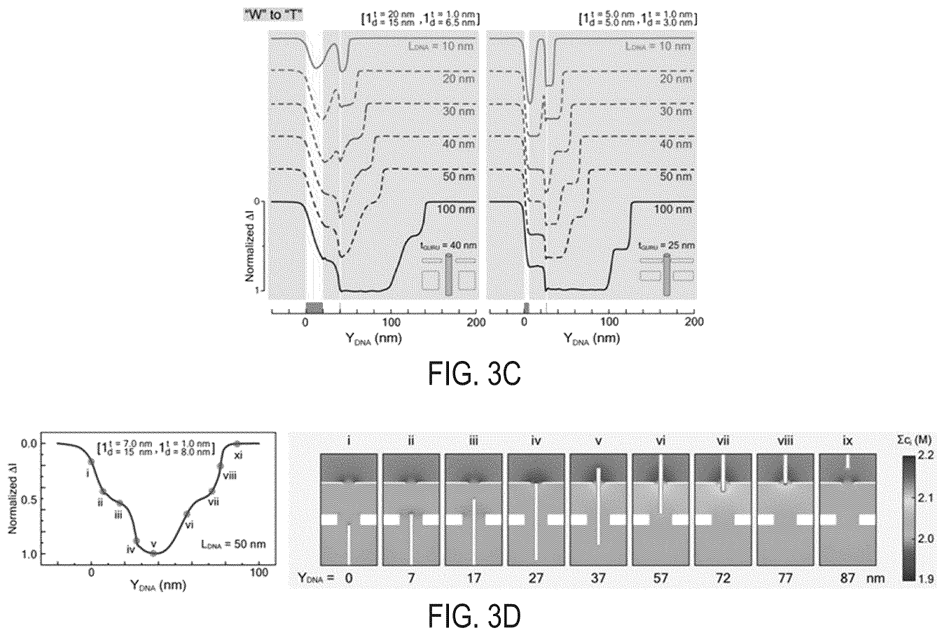

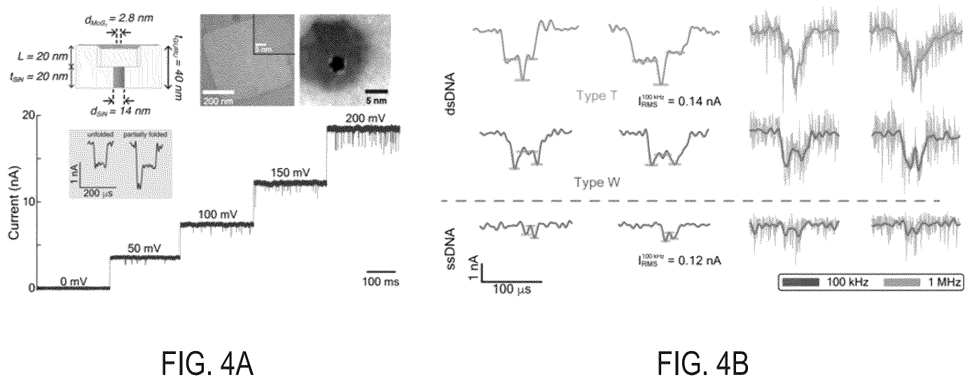

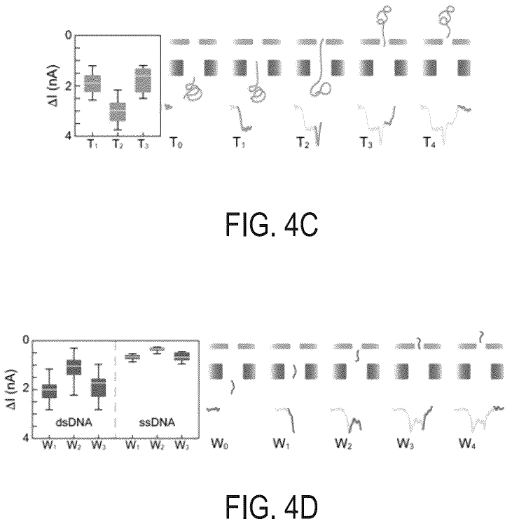

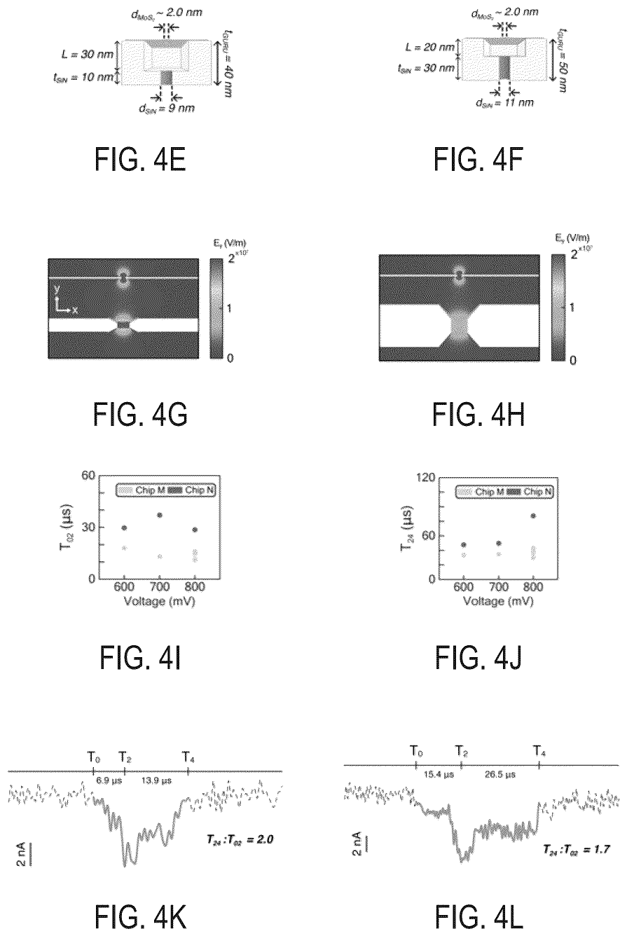

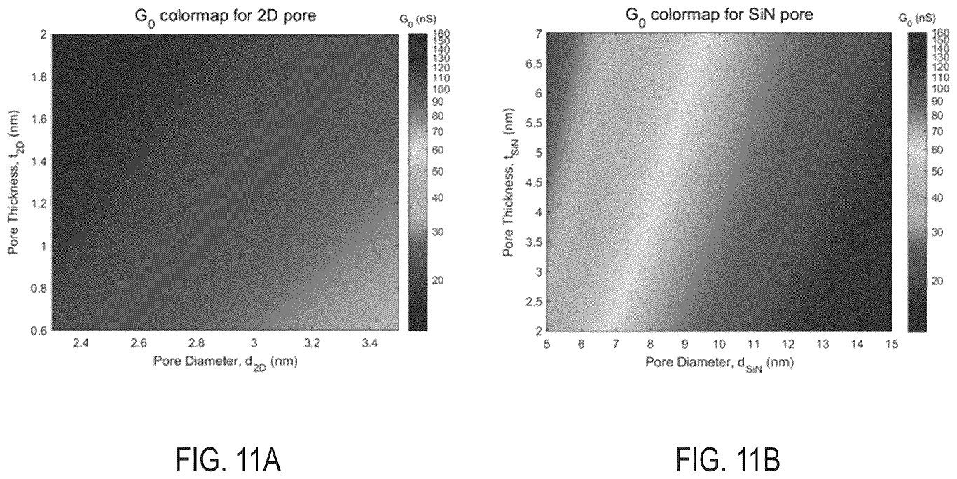

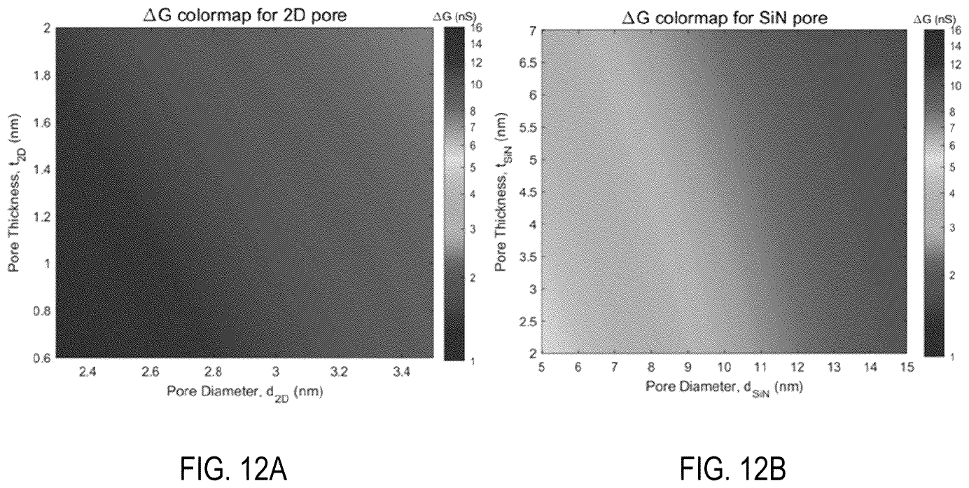

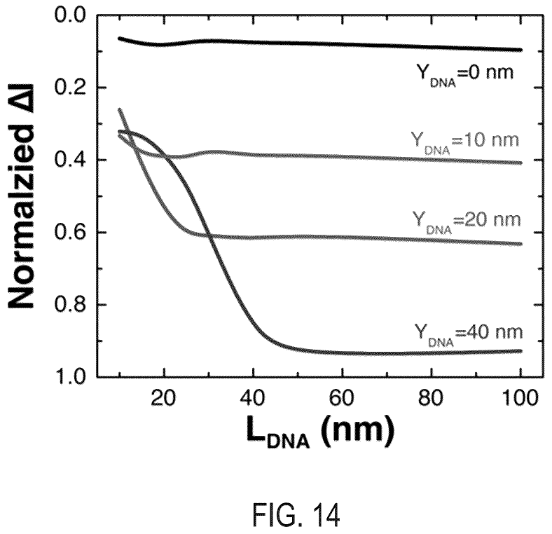

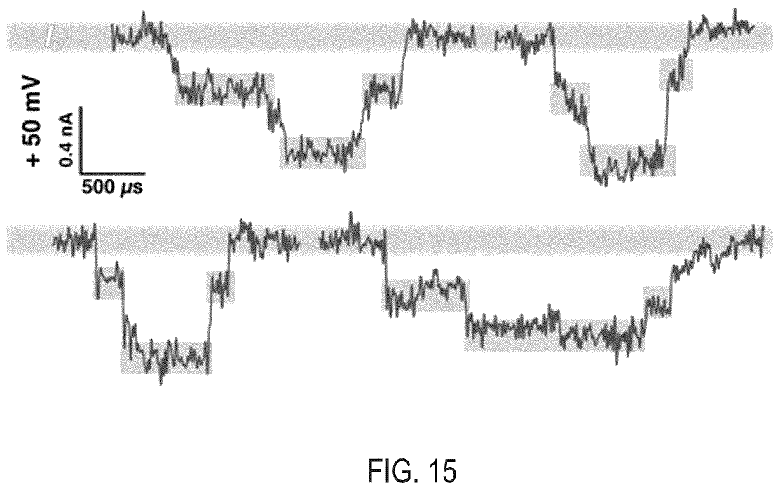

Distinctive translocating ionic signal pattern arises due to coupling electric fields. Current simulations in open pores and dsDNA blockade at varying y-positions. Y_{DNA}, are performed for different configurations (FIG. 3A-3D). For decoupled pores, two events occur independently. When G_{2D}<<G_{GURU} and pores are coupled as in [6,1] (FIGS. 2A-2E), U-events arise merely from the top 2D layer. However, in the regime of G_{2D}˜G_{GURU}, when limiting N=M=1, we observe interference from both pores and blockade patterns resembling variations of “W” and “T” shapes vs. dsDNA positions (FIG. 3B). As the coupled electric field distributes across two pores, the blockade in one pore interferes with that in the other. In this simulation, these shapes are mostly symmetric (T₁˜T₃) when the individual current blockades of dsDNA are similar, which reflects nontrivial information on GURU structure. For example, the simulation result demonstrates the influence of voltage on the asymmetry of T₁ and T₃, which may result from the ions depleted zone in front of the DNA when DNA occupies only the SiN pore and the accumulation of ions in the trench region when DNA occupies only the 2D pore (FIGS. 11A-11B-13A-13D). We illustrate, for d_{2D}=3 nm to 14 nm, how signals evolve from U to W and U to T, for L_{DNA}<and >t_{GURU}, respectively, by fixing other parameters. The range of d_{2D}, corresponding to d_{2D}/d_{SiN}=0.20 to 0.93, is determined based on the open-pore conductances and modeling for the data shown in FIG. 4A. Signal sublevels are marked as T₁₋₃ for T-events and W₁₋₃ for W-events. The minimum and maximum blockade sublevels are W₂ (dsDNA in the trench), and T₂ (cross-layer dsDNA occupancy), respectively. With the coupling effect, T₂ depth is not the sum of T₁ and T₃ (single-layer dsDNA occupancy), and T₁ can be >,˜, or <T₃ (FIGS. 4C-4D). FIG. 3C displays simulated shape evolution as L_{DNA} increases by 10 nm for two sets of geometric parameters in [1, 1]. W-events occur where L_{DNA}<t_{GURU}, while T-events emerge where L_{DNA}>t_{GURU}. The results indicate signals are sensitive to nm-size changes in L_{DNA}, in principle allowing accurate single-molecule sizing. From G (at fixed Y_{DNA}) VS. L_{DNA} (FIG. 14), we estimate ΔG/ΔL_{DNA}˜2-9 nS/kbp. Moreover, sublevel time stamp analysis allows real-time label-free DNA position-tracking with nm accuracy.

The detection of dsDNA position and travel time with [1,1] configuration is possible by advancements in, 1) temporal resolution (1 μs), permitting short feature identification, and 2) exquisite nanofabrication, permitting in-series assembly of atomically thin pores. Provided reported translocating velocity⁹, dsDNA would travel L=20 nm between layers in ˜7-70 μs. This is possible for detection, and we demonstrate such a possibility to guide and track unmodified DNA travel across a ˜20 nm distance and with ˜1-μs time resolution. This capability represents an improvement of more than one order of magnitude in the smaller distance and also translocation time between tracking markers shown previously for labeled dsDNA⁹.

### Experimental Observations of T- and W-event Signals in [1,1]

The experimental results of Chip L, [1,1], are shown. The as-fabricated TEM-estimated diameters were d_{2D}(TEM)=2.8±0.7 nm, and d_{SiN}(TEM)=14.0±0.9 nm, respectively (FIG. 4A). Detected current-time traces and event shapes resemble simulations in FIGS. 3A-3D and starkly contrast to the typical U-shape events produced by small-diameter single pores. These short signal features from label-free DNA passing through two pores are distinguishable at high bandwidth (root-mean-square noise Iᵣₘₛ^{1MHz}˜0.5 nA). The ultrafast detection comes at the price of sacrificing low-magnitude signals and distorting the distribution towards deeper events, presenting an everlasting experimental tug-of-war between low-noise and high-time-resolution. Here, the latter is prioritized to detect DNA passage through both pores. Besides, orders of magnitude faster detection is another advantage over protein pores in applications where minimizing measurement time to seconds is important. We note that the applied voltage and the concentration of salt solutions were selected and limited not by the device but by the amplifier current limit of 18 nA.

For small L_{DNA} (200-nt ssDNA), most detected events are W-events; T-events were extremely rare (<0.6%) (FIG. 4B). Although short ssDNA could, in principle, stretch across pores (L_{DNA}˜126 nm>L), this is deemed unlikely since Lₚ˜1 nm<<L²⁴. Despite the low SNR, W-events from ssDNA can still be resolved with a magnitude about half that for dsDNA W-events using the same device. Further SNR improvement using lower-noise substrates, glass or smaller pore diameters for signal enhancement is required for better ssDNA detection.

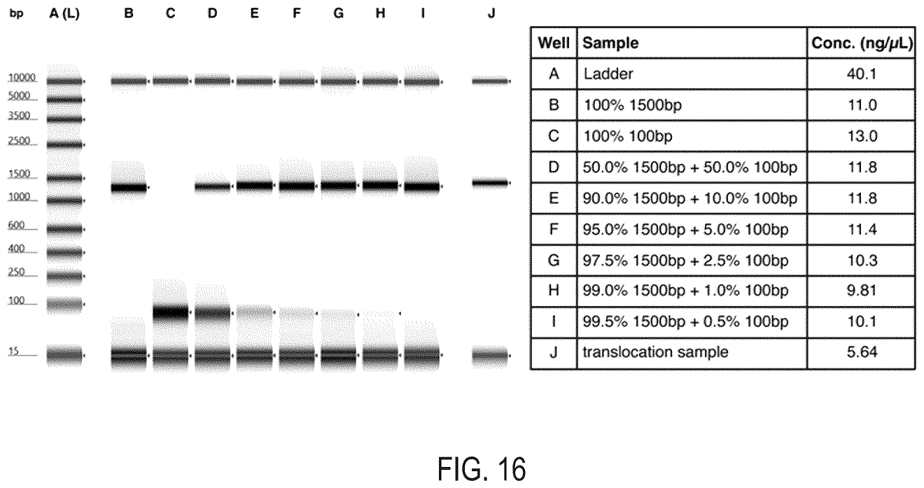

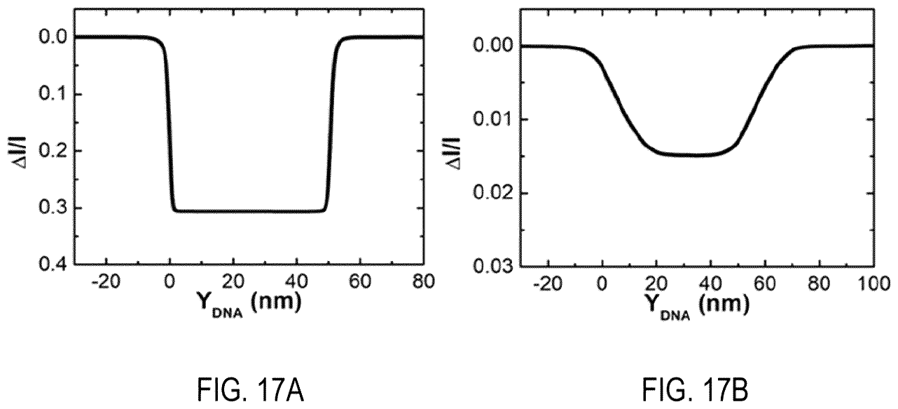

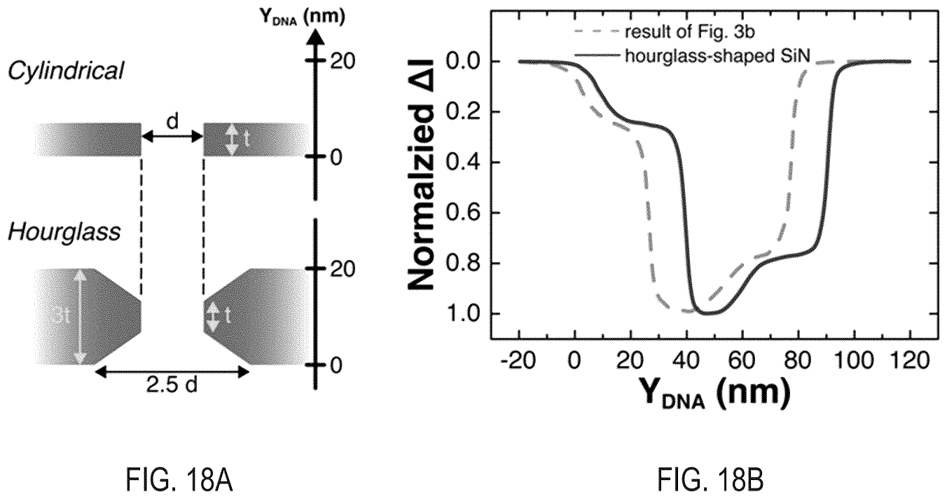

The translocation measurements for 1500-bp dsDNA (L_{DNA}˜450 nm) are displayed in FIGS. 4C-4D. T-events are detected as expected for L_{DNA}>>t_{GURU}, and dsDNA translocate like a rod, with Lₚ=35-50 nm>L^{24,25}. Selected long events at 50 mV (FIG. 15) show a timescale similar to the polymer relaxation (Zimm) time²⁶. Rare W-events (˜5%) were detected with a shorter dwell time than T-events. Similar depths of W_{1,3}˜T_{1,3} hint at possible fragmented dsDNA contamination (FIG. 4B). We performed gel electrophoresis on the measured sample (FIG. 16). Average sizing signals of ˜1300-bp dsDNA were detected from samples containing 1500-bp dsDNA, without clear indications of fragmented pieces. Yet, the tool limitation was validated using mixtures of pure samples (1500-bp and 100-bp dsDNA. 0.5-50%), where the controlled mix of 1% 100-bp dsDNA best-matched electrophoresis signals of the measured sample detecting “W-events.” Our data reveal the unprecedented ability and sensitivity of GURU devices to discriminate low-concentration (<1%) analytes within the same sample. In contrast, a single-pore sensor produces unanimous U-events (FIG. 4A, inset) with uniform event depths but wide dwell time distributions²⁷. Others factor that may affect event shapes are discussed herein (FIGS. 17-19).

Tailored t_{SiN} and/or t_{GURU} can be employed to manipulate coupled electric fields and attain localized weakening of E_{y} upon translocations. FIGS. 4E and 4F showcase two GURU devices, Chips M and N, with t_{SiN}=10±1 and 30±1 nm, d_{SiN}(TEM)=9 and 11 nm, L=30±1 and 20±1 nm, while fixing MoS₂ pores geometries (photos in SI). Simulated electric field profiles at 800 mV and 3M KCl are shown in FIGS. 4G and 4H. The corresponding values of E_{y} in MoS₂ pores are designed to be at similar value, −2.53 and −2.47 (×10⁸ V/m), while in SiN pores are about 2 times of differences, −1.25 and −0.68 (×10⁷ V/m), which arises from the differences in geometrical design. The hourglass nanopore geometry¹³ is considered. Translocation measurement is conducted with 3M KCl, 200-bp dsDNA at various voltages (Table S2). To probe translocation dynamics between SiN pore and MoS₂ pore, we analyze the dwell time before and after dsDNA enters the MoS₂ layer. Since the contour length of 200-bp dsDNA is longer than t_{GURU} for both devices, T-events dominate the event populations. With a weaker E_{y} in SiN layer, we observe a longer dwell time in T₀₂, T₂₄ and T₀₄ at fixed voltages (FIGS. 4I and 4J). This indicates that the presence of a weaker electric field from SiN layer can effectively prolong the dwell time. We also observe a correlation between the ratio T₂₄:T₀₂ and the measured devices. With t_{SiN}=10 nm, the ratio of T₂₄:₀₂ is ˜1.8, while the ratio for the device with t_{SiN}=30 nm has an average of ˜2.2. FIGS. 4K and 4L are sample events from both devices at 800 mV. 10 MHz bandwidth, Bessel filtered at 1 MHz, with the ratio T₂₄:T₀₂=2.0 and 1.7.

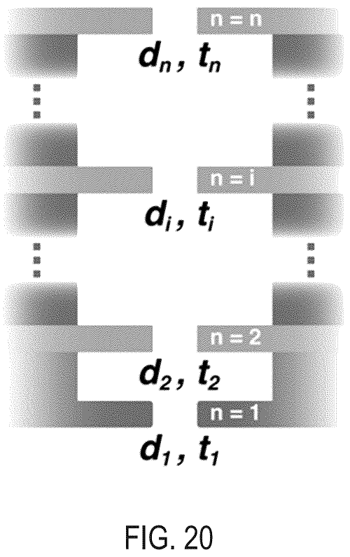

In summary, coupled nanopores represent a broad new concept opening possibilities in molecule transport and readout within a vast parameter space without labels or enzymes (FIG. 20). Rather than modifying molecules, here we aimed to exploit superior nanofabrication capabilities to imprint nanostructures enabling advanced electromechanical control and detection. Interesting finds include biphasic (combined “down” and “up”) events for small d_{SiN} and high polymer charges. GURU platforms guide molecules with constrained trajectories and can accommodate different 2D materials, possibly, lipid bilayer membranes and protein pores. Their reusability and scalability demonstrate cost-efficiency. 2D multilayers may be also incorporated while improving SNR and temporal resolution to 100 ns¹⁴. Together with passive-electromechanical control shown here, GURU platforms could be augmented with active multi-channel restraints to electrically control individual nanopore readout for the next generation of advanced solid-state nanopore systems.

### Materials and Methods

GURU Fabrication in SiN Layer. To fabricate the trench region within the GURU devices, the 5-mm chips are first made through traditional microfabrication methods^{28,29} to create a suspended SiN membrane window, from bottom to top, a 500-μm silicon substrate, a 5-μm thermal SiO₂, and a 40-nm (or 120-nm) low-stress LPCVD SiN. The chip is first spin-coated with C4 PMMA at 4000 rpm for 60 seconds, followed by a 10-minute baking at 180° C. A pair of two-square pattern, 400 by 400 nm², is exposed onto the resist layer (Elionix ELS-7500EX). The resist is developed in 1:3 v/v MIBK(Methyl isobutyl ketone):IPA(Isopropyl alcohol) solution for 60 seconds, and rinsed in IPA for 2 minutes. Combining reactive ion etching (RIE), with trifluoromethane and oxygen (CHF₃/O₂) at a rate of ˜1 nm/s, we can remove SiN within the patterned area^{28,29} to the desired trench depth, L. Prior to fabricate nanopores in the SiN layer, boiling piranha solution (1:3 v/v H₂O₂:H₂SO₄) is used for 10-15 minutes to remove the leftover resist and any organic contamination.

There are two methods we utilized in this work to fabricate nanopores in the SiN layer, TEM-drilling and a combination of EBL and RIE. For in-situ room temperature TEM drilling, we operate at 200 kV for high-resolution TEM mode (JEOL F200). This operating mode allows us to locate the thinned square region and fabricate one nanopore at a time to desired numbers³⁰. On the other hand, to quickly fabricate a significant number of nanopores onto the SiN layer, 1:2 dilution of ZEP520A:Anisole is spin-coated at 4000 rpm for 40 seconds, followed by 2-minute baking at 180° C. Array patterning is exposed onto the resist layer (Elionix ELS-7500EX) with a shot pitch of 150 nm³⁰. The resist is then developed in o-xylene for 70 seconds and IPA for 30 seconds. Controlling the etching time in RIE steps, we can fabricate nanopores only in the previously thinned square area, instead of creating a nanoporous SiN membrane. The resist is then stripped off by placing the membrane in heated N-methyl-2-pyrrolidone (NMP) at 60° C. for 3 hours and then rinsed with IPA. Before transferring 2D layers onto the device, another round of 10-minute piranha cleaning is performed.

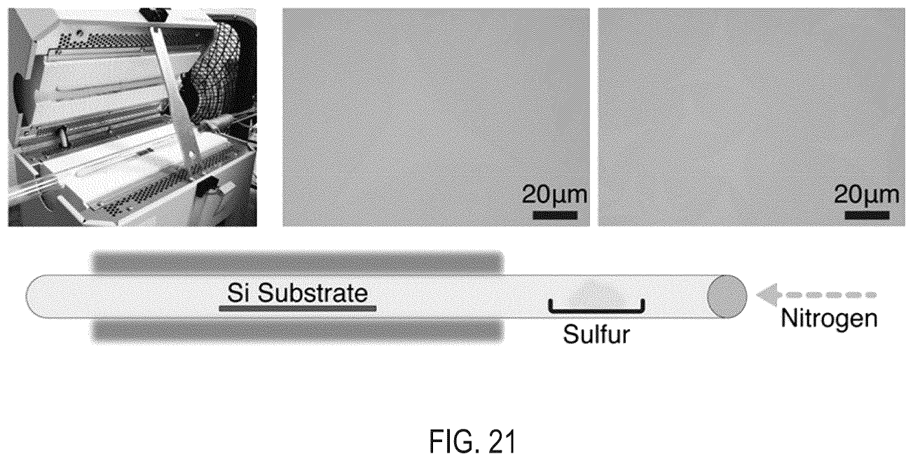

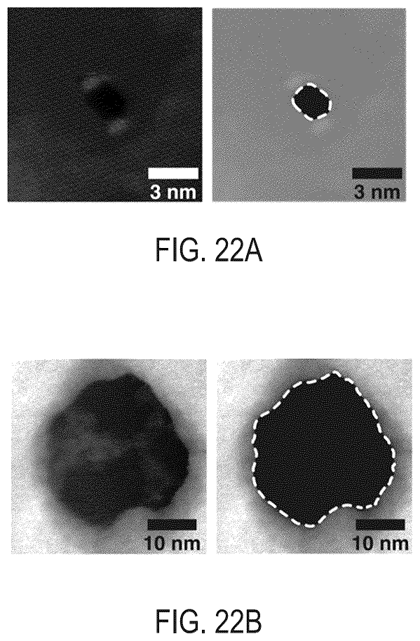

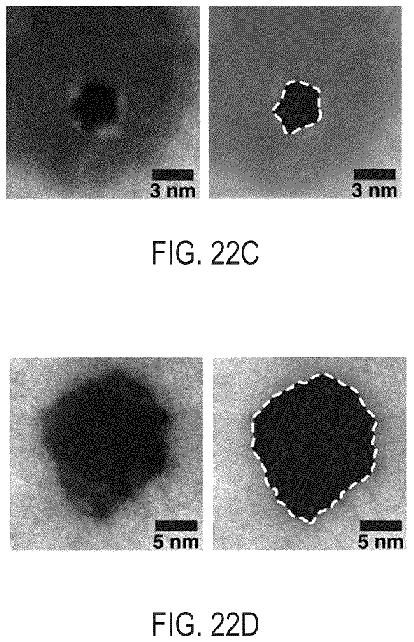

2D MoS₂ Growth, Device Integration, and Nanopore Drilling. Monolayer MoS₂ flakes were grown via chemical vapor deposition (CVD), schematic shown in FIG. 21, following a previously described process³¹. Solutions of ammonium heptamoly bdate tetrahydrate (9 mM) and sodium cholate (23 mM) in deionized water were spun-coated onto a piranha cleaned Si/SiO₂ substrate (300 nm SiO₂). The substrate was placed in a 1-inch tubular furnace (Thermo Scientific Lindberg/Blue M), and 150 mg of sulfur was placed on a second SiO₂/Si substrate 22 cm upstream. The furnace was flushed with N₂ gas flow (1000 sccm) for 10 minutes, after which the growth substrate was heated at a rate of 70° C./min and kept at 750° C. for 15 minutes under 400 sccm N₂ gas flow. During growth, the sulfur was kept at a temperature of 180° C. After growth, the samples were rapidly cooled by turning off and opening the furnace.

After the SiN scaffold fabrication process and piranha cleaning, monolayer MoS₂ flakes were transferred onto the GURU devices following a wet transfer process³². After CVD growth, a piece of SiO₂/Si substrate coated with MoS₂ flakes was spun-coated with PMMA and placed on a 1 M KOH solution to etch the underlying SiO₂. The floating PMMA film with MoS₂ flakes was then washed by transferring it to a large volume of DI water before being scooped up and transferred onto the device and positioned under an optical microscope so that a single MoS₂ flake fully covered the scaffold structure. After drying for one hour in the air, the PMMA was removed with acetone and subsequent rapid thermal annealing (RTA) in Ar:H₂ mixture.

2D MoS₂ imaging and nanopore drilling were performed on a Cs-corrected JEOL NEOARM scanning transmission electron microscope (STEM) operating at 80 kV. The nanopores were drilled by scanning the electron probe over a selected area ranging from 1 to 9 nm² for up to 10 seconds. With this technique, we can create nanopores with tailored sizes in a controlled manner.

Device Wetting and Storage Procedure. The wetting procedure begins with mixing equal amount of EtOH:H₂O (v/v=1/1) solutions (HPLC grade ethanol from Fisher Chemical and DI water) at room temperature. This wetting solution is then undergoing vacuum degassing for 30 minutes. Right after drilling the nanopore in the 2D layer, the GURU device is immersed in the degassed solution for 30-60 minutes.

Determining Nanopore Geometries and Error Estimation in Thickness. The GURU membrane thickness, t_{SiN}, is measured by ellipsometry after fabrication. The initial pore diameters, d, are measured from TEM images, corresponding to the smallest constriction region within the pore, error is determined as discussed herein (FIG. 21). The inter-layer separation, L, is calibrated by RIE etching time (˜1 nm/s) and the starting membrane thickness. We estimate the error in L to originate from fluctuations in the starting deposition thickness (˜40 nm) and the error in etching time (˜1 s) performed by the user. This translates to an estimated error in L. Based on this we quote in the main text L=20±1 nm and t_{GURU}=40±3 nm.

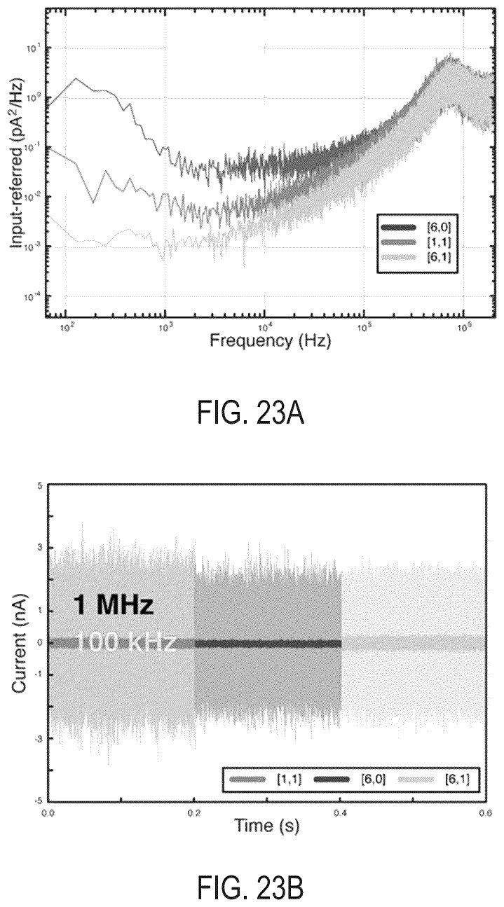

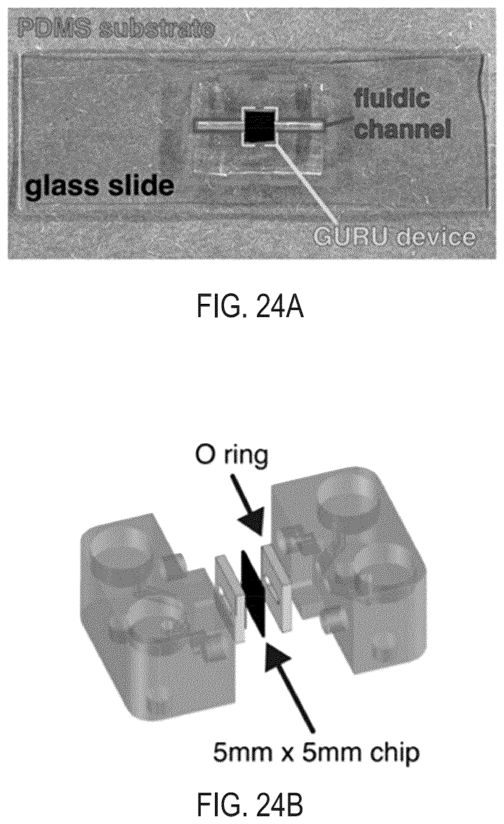

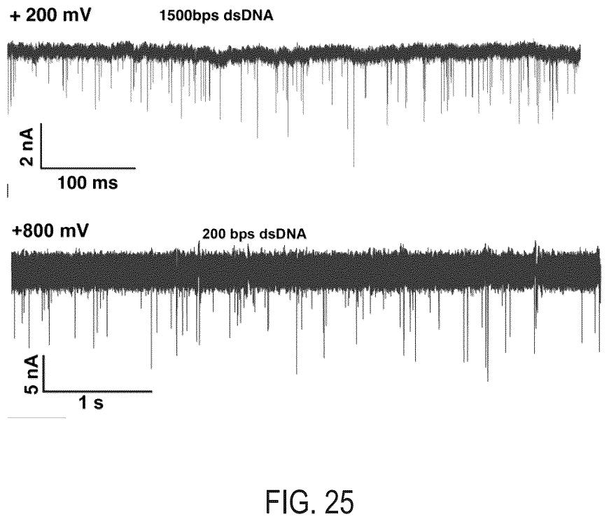

Ionic Measurements and DNA Sample. Recording Bandwidth of 1 MHz VC100 amplifier (Chimera Instruments, New York, NY) and that of 10 MHz amplifier (Elements, SRL) are utilized to read ionic signals by applying an external bias voltage via a two-terminal set of Ag/AgCl electrodes (see FIG. 23 for noise performance discussion). Two types of nanopore flow cell are used in this work to host the GURU devices. Conventional one-piece PDMS flow cells³³ are used for devices measured in FIG. 2, where the devices are glued to the flow cell with silicone elastomers (FIG. 24A). The other customized two-parts PMMA flow cell (FIG. 24B) is used to house the device measured in FIG. 4. Within this design, two complementary PMMA parts are manufactured with a cavity for the GURU device, and fluidic channels connect reservoirs with a volume between 60 to 70 μL. To assemble and secure the GURU device, the device is sandwiched between two silicon gaskets, labelled as O-rings, which are subsequently compressed by the PMMA plastic parts. Some examples of current traces are given in FIG. 25.

The DNA samples used for ionic measurements are 1500-bp dsDNA (200 nM, NoLimits, ThermoFisher Scientific), 200-bp dsDNA, and 90-nt (FIGS. 2D and 2E) and 200-nt (FIG. 4A) ssDNA (200 nM, IDT), and authenticated by the company. Samples are prepared in the TE buffer (10 mM Tris, 1 mM EDTA), 1M and 3M KCl electrolyte solution (pH=8.0) at room temperature. Oligo sequence for 90-nt ssDNA is, GCGTAATACGACTCACTATAGTCTTTGCAGCACCGACACC

TGAACTTCCACCCTTCTTTCAAGTCATGTTCTTTAGTGAGGGTTAA TTCG; and, for 200-nt ssDNA is,

### GCGTAATACGACTCACTATAGTCTTTGCAGCACCGACACCTGAACTTCC

ACCCTTCTTTCAAGTCATGTTCTTTAGTGAGGGTTAATTCGCCCCC CCCCCCCCCCCCCCCGCGTAATACGACTCACTATAGTCTTTGCAGCACCGACACC TGAACTTCCACCCTTCTTTCAGTCATGTTCTTTAGTGAGGGTTAATTCG.

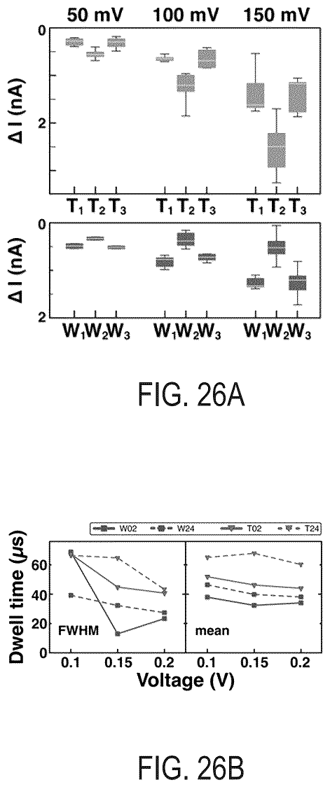

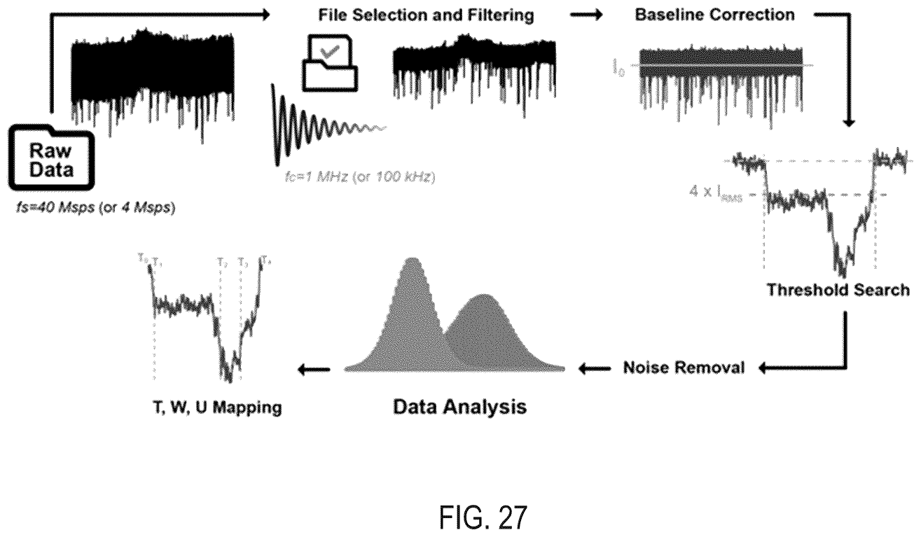

Data Analysis Details. Event identification and characterization such as in FIG. 4 and FIG. 26 are completed in a two-step process. Baseline correction and threshold detection is first used to extract events from filtered current traces (FIG. 27). Event types and features are then inspected selected manually for subsequent analysis. We note that the typical characterization used in the solid-state nanopore field defining the mean event depths and durations is not particularly meaningful and informative for data containing characteristic T-like and W-like event patterns. For event analysis in FIG. 4, we consider events longer than 10 μs (or 1 μs), corresponding to the digital Bessel filter we applied to the cut-off frequency, 100 kHz (or 1 MHz). Due to bandwidth limitation and time resolution limitation to ˜1 μs, the nanopore data analysis is inherently biased towards events longer than this time limit and shorter events are not detected. Event characterization tools and an interactive GUI for feature selection can be found at github.com/joshualchen/Clampfit.

### References

1. Xue, L. et al. Solid-state nanopore sensors. Nat Rev Mater 5, 931-951 (2020).
2. Manrao, E. A. et al. Reading DNA at single-nucleotide resolution with a mutant MspA nanopore and phi29 DNA polymerase. Nat Biotechnol 30, 349-353 (2012).
3. Danda, G. & Drndić, M. Two-dimensional nanopores and nanoporous membranes for ion and molecule transport. Current Opinion in Biotechnology 55, 124-133 (2019).
4. Healy, K., Schiedt, B., Morrison, I. P. & Morrison, A. P. Nanomedicine 2, 875 (2007).
5. Branton, D. et al. The potential and challenges of nanopore sequencing. Nat Biotechnol 26, 1146-1153 (2008).
6. Dekker, C. Solid-state nanopores. Nature Nanotech 2, 209-215 (2007).
7. Deamer, D. W. & Akeson, M. Nanopores and nucleic acids: prospects for ultrarapid sequencing. Trends in Biotechnology 18, 147-151 (2000).
8. Chen, K., Gularek, F., Liu, B., Weinhold, E. & Keyser, U. F. Electrical DNA Sequence Mapping Using Oligodeoxynucleotide Labels and Nanopores. ACS Nano 15, 2679-2685 (2021).
9. Chen, K. et al. Dynamics of driven polymer transport through a nanopore. Nat. Phys. 17, 1043-1049 (2021).
10. Niedzwiecki, D. J. et al. Devices for Nanoscale Guiding of DNA through a 2D Nanopore. ACS Sens. 6, 2534-2545 (2021).
11. Merchant, C. A. et al. DNA Translocation through Graphene Nanopores. Nano Lett. 10, 2915-2921 (2010).
12. Masih Das, P. et al. Atomic-scale patterning in two-dimensional van der waals superlattices. Nanotechnology (2019) doi: 10.1088/1361-6528/ab596c.
13. Rodríguez-Manzo, J. A., Puster, M., Nicolaï, A., Meunier, V. & Drndić, M. DNA Translocation in Nanometer Thick Silicon Nanopores. ACS Nano 9, 6555-6564 (2015).
14. Chien, C.-C., Shekar, S., Niedzwiecki, D. J., Shepard, K. L. & Drndić, M. Single-Stranded DNA Translocation Recordings through Solid-State Nanopores on Glass Chips at 10 MHz Measurement Bandwidth. ACS Nano 13, 10545-10554 (2019).
15. Fischbein, M. D. & Drndić, M. Electron beam nanosculpting of suspended graphene sheets. Appl. Phys. Lett. 93, 113107 (2008).
16. Thiruraman, J. P., Masih Das, P. & Drndić, M. Stochastic Ionic Transport in Single Atomic Zero-Dimensional Pores. ACS Nano 14, 11831-11845 (2020).
17. Masih Das, P. et al. Controlled Sculpture of Black Phosphorus Nanoribbons. ACS Nano 10, 5687-5695 (2016).
18. Pedone, D., Langecker, M., Abstreiter, G. & Rant, U. A Pore—Cavity—Pore Device to Trap and Investigate Single Nanoparticles and DNA Molecules in a Femtoliter Compartment: Confined Diffusion and Narrow Escape. Nano Lett. 11, 1561-1567 (2011).
19. Langecker, M., Pedone, D., Simmel, F. C. & Rant, U. Electrophoretic Time-of-Flight Measurements of Single DNA Molecules with Two Stacked Nanopores. Nano Lett. 11, 5002-5007 (2011).
20. Briggs, K. et al. DNA Translocations through Nanopores under Nanoscale Preconfinement. Nano Lett. 18, 660-668 (2018).
21. Smeets, R. M. M. et al. Salt Dependence of Ion Transport and DNA Translocation through Solid-State Nanopores. Nano Lett. 6, 89-95 (2006).
22. Lastra, L. S., Bandara, Y. M. N. D. Y., Nguyen, M., Farajpour, N. & Freedman, K. J. On the origins of conductive pulse sensing inside a nanopore. Nat Commun 13, 2186 (2022).
23. Shekar, S. et al. Measurement of DNA Translocation Dynamics in a Solid-State Nanopore at 100 ns Temporal Resolution. Nano Lett. 16, 4483-4489 (2016).
24. Kowalczyk, S. W., Tuijtel, M. W., Donkers, S. P. & Dekker, C. Unraveling Single-Stranded DNA in a Solid-State Nanopore. Nano Lett. 10, 1414-1420 (2010).
25. Brinkers, S., Dietrich, H. R. C., de Groote, F. H., Young, I. T. & Rieger, B. The persistence length of double stranded DNA determined using dark field tethered particle motion. The Journal of Chemical Physics 130, 215105 (2009).
26. Bell, N. A. W., Chen, K., Ghosal, S., Ricci, M. & Keyser, U. F. Asymmetric dynamics of DNA entering and exiting a strongly confining nanopore. Nat Commun 8, 380 (2017).
27. Carson, S., Wilson, J., Aksimentiev, A. & Wanunu, M. Smooth DNA Transport through a Narrowed Pore Geometry. Biophysical Journal 107, 2381-2393 (2014).
28. Wanunu, M. et al. Rapid electronic detection of probe-specific microRNAs using thin nanopore sensors. Nature Nanotech 5, 807-814 (2010).
29. Venta, K. et al. Differentiation of Short, Single-Stranded DNA Homopolymers in Solid-State Nanopores. ACS Nano 7, 4629-4636 (2013).
30. Chou, Y.-C., Masih Das, P., Monos, D. S. & Dmdić, M. Lifetime and Stability of Silicon Nitride Nanopores and Nanopore Arrays for Ionic Measurements. ACS Nano 14, 6715-6728 (2020).
31. Parkin, W. M. et al. Raman Shifts in Electron-Irradiated Monolayer MoS2. ACS Nano 10, 4134 (2016).
32. Mlack, J. T. et al. Transfer of monolayer TMD WS2 and Raman study of substrate effects. Sci Rep 7, 43037 (2017).
33. Balan, A. et al. Improving Signal-to-Noise Performance for DNA Translocation in Solid-State Nanopores at MHz Bandwidths. Nano Lett. 14, 7215-7220 (2014).

### Additional Disclosure

### Discussion: Finite Element Analysis in 3D

Simulations are based on finite element analysis. The ion transport in the two-layers is modeled by modified Poisson-Nernst-Planck and Navier-Stokes equations with the associated boundary conditions, ^{1,2} as following,

$$- {ε}_{f} {∇}^{2} ϕ = F {\sum }_{i = 1}^{2} {z}_{i} {c}_{i} \qquad ( S1 )$$

$$∇ \cdot {J}_{i} = ∇ \cdot [ - {D}_{i} ∇ {c}_{i} - {D}_{i} \frac{{z}_{i} F}{RT} {c}_{i} ∇ ϕ - {a}_{i}^{3} {D}_{i} \frac{{c}_{i} {\sum }_{i = 1}^{2} ∇ {c}_{i}}{1 - {\sum }_{i = 1}^{2} {a}_{i}^{3} {c}_{i}} + {uc}_{i} ] = 0 \qquad ( S2 )$$

$$- ∇ p + μ {∇}^{2} u - F {\sum }_{i = 1}^{2} {z}_{i} {c}_{i} ∇ ϕ = 0 \qquad ( S3 )$$

Here ϕ is electric potential; cᵢ, Dᵢ, zᵢ, and Jᵢ are the concentration, diffusivity, valence, and flux of i^{th} ionic species, respectively; _{εf}, F, R, and T are the fluid permittivity, Faraday constant, gas constant, and absolute temperature, respectively; u, p, and μ are the fluid velocity, hydrodynamic pressure, and dynamic fluid viscosity, respectively. The liquid phase is assumed as an incompressible Newtonian fluid. aᵢ denotes the effective ion size of i^{th} ionic species. The inclusion of a correction term (third term in Eq. (S1)) accounts for steric effects of ions. We assume both potassium and chloride ions have the same ionic size of 0.6 nm ¹. This continuum-based theory is valid for describing ion transport when scale is larger than 1 nm. All simulations were carried out with COMSOL Multiphysics (version 5.6) and 3D geometry was built in.

As illustrated in FIG. 5, one 2D pore with 3-8 nm diameters and 1 nm thickness is on the top of the trench, while one (six) 15-nm-diameter SiN nanopore(s) with 5-20 nm thickness is (are) on the bottom of the trench. The trench is 400 nm in length and width, and 20 nm in height. A voltage bias is applied to the top reservoir while the bottom is grounded. Both the surfaces (2D and SiN pores) are negatively charged with −0.02 C/m^{2 3,4}. The ionic current is calculated by integrating the ionic flux over the reservoir surface. DNA is assumed as a charged rod of −0.03 C/m², and is driven along the y-axis from the SiN pore to the 2D pore. Take 20-nm-thick SiN as an example, Y_{DNA}=0 and Y_{DNA}=40 nm are the position when DNA enters the SiN pore and 2D pore, respectively.

### 1.1. Electrophoretic Velocity of DNA

The translocation velocity can be modeled by imposing force balance on the DNA, as described previously ^{5,6}. We assume that DNA moves straight along the central axis. The contributions of electric force and hydrodynamic force are based on the integrations of Maxwell tensor and hydrodynamic stress tensor over the rod surface, which can be expressed as,

$${F}_{e} = {\int }_{{Ω}_{p}} ( ε EE - \frac{1}{2} {E}^{2} I ) \cdot n d {Ω}_{p} \qquad ( S4 )$$

$${F}_{h} = {\int }_{{Ω}_{p}} ( - pI + η [ ∇ u + {( ∇ u )}^{T} ] ) \cdot n d {Ω}_{p} \qquad ( S5 )$$

Because [1,1] is axial symmetric, 2D cylindrical coordinates are adopted to save computational cost. Assuming pseudo-steady state, the net force acting on the DNA along the pore axis vanishes ⁵:

$${\int }_{{Ω}_{p}} ε ( \frac{\partial ϕ}{\partial r} \frac{\partial ϕ}{\partial z} {n}_{r} + \frac{1}{2} [ {( \frac{\partial ϕ}{\partial z} )}^{2} - {( \frac{\partial ϕ}{\partial r} )}^{2} ] {n}_{z} ) d {Ω}_{p} + {\int }_{{Ω}_{p}} [ {n}_{r} η ( \frac{\partial {u}_{r}}{\partial z} + \frac{\partial {u}_{z}}{\partial r} ) + {n}_{z} ( - p + 2 η \frac{\partial {u}_{z}}{\partial z} ) ] d {Ω}_{p} = ( \qquad ( S16 )$$

Then the velocity is determined by solving coupled Eqs. (S1)-(S3) and (S6).

### Discussion: The Scalability and Reusability of GURU Device

Multiple thinned squares could be placed in the same device. In FIG. 10A, we demonstrate a potential wafer-scale integration with multiple trench patterning. After ionic current measurements, significant 2D MoS₂ pore expansion can be observed upon AC-TEM imaging. The SiN part of the GURU device can then be reused after a cleaning procedure consisting of 10 minutes in piranha solution, followed by a 90-minute RTA treatment. This step removed the 2D MoS₂ layer entirely, as well as majority of other contaminants. A new set of 2D MoS₂ flakes can then be transferred onto the same GURU platform for another round of 2D pore drilling and ionic current measurements. In between measurements, devices are stored under vacuum till next transfer step takes place.

One device was reused up to 4 times, as demonstrated in this work (FIG. 2A). After first round of measurement, the device was cleaned and a conformal HfO₂ layer is deposited via Cambridge Nanotech S200 ALD system⁷, in order to further decrease the pore diameters of SiN layer. The deposition of HfO₂ can be seen clearly under DF mode in white circles around the pore edges. It was used to test the wetting ability of the device, and no translocation measurement was conducted with HfO₂-coated samples (˜3 nm). Consecutive TEM images (top of FIG. 10B), and optical images of each new round of 2D materials transfer (bottom of FIG. 10B) of the same device were taken. Visible contamination grew as the device was reused over time.

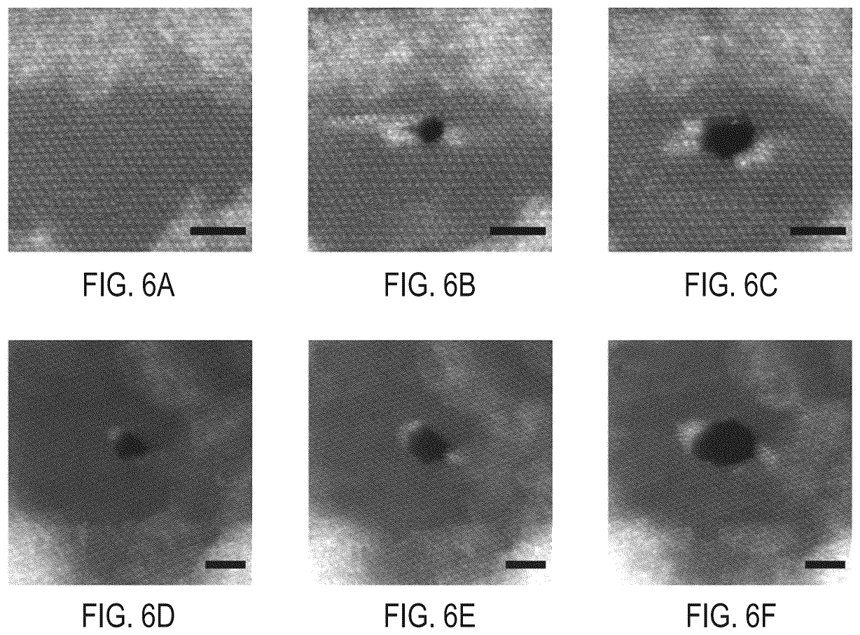

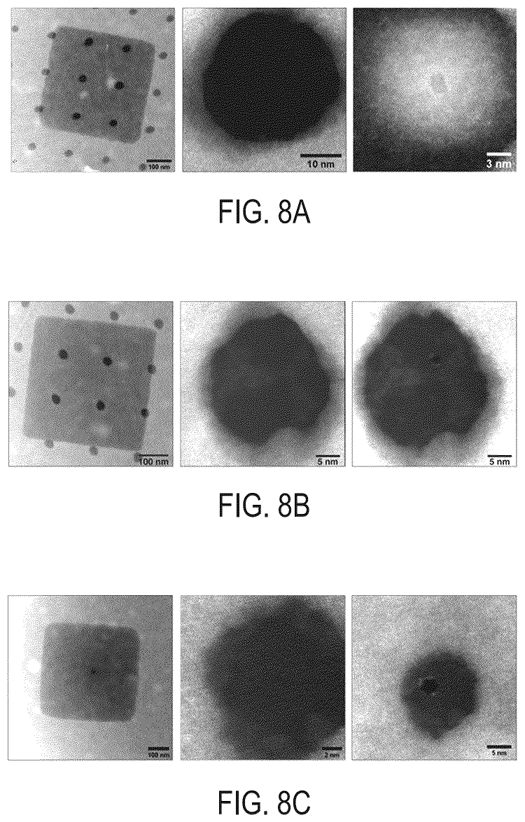

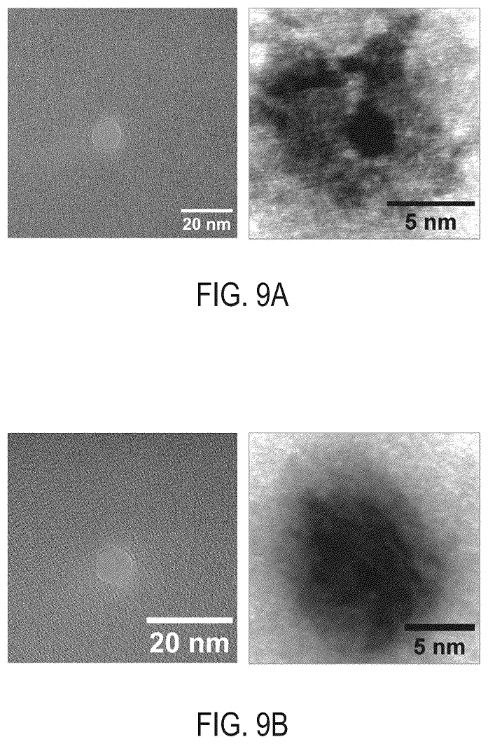

TABLE S1
Index of TEM images for GURU devices with various configurations.

| Chip # | Configuration | |
|---|---|---|
| A | [1, 0] | FIG. 2a |
| B | [2, 0] | FIG. 2a |
| | [2, 1] | FIG. 7A, B |
| C | [3, 0] | FIG. 2a |
| D | [9, 0] | FIG. 2a and 20 |
| | [9, 1] | FIG. 2a |
| E | [4, 0] | FIG. 2a |
| F | [6, 1] | FIG. 6A, B, C |
| G | [6, 1] | FIG. 6D, E, F |
| H | [6, 1] | FIG. 7C, D |
| I | [9, 1] | FIG. 7E, F |
| J | [6, 1] | FIGs. 2b, d and 8A |
| | [6, 0] | FIG. 2d |
| K | [4, 1] | FIGs. 2e and 8B |
| L | [1, 1] | FIG. 4a |
| M | [1, 1] | FIGs. 4e and 9A |
| N | [1, 1] | FIGs. 4f and 9B |

### Discussion: Analytical Expressions for the Current and Current Blockade and Discussion of Independent Pores vs. Coupling Effects

### Individual Pores

In addition to the 3D finite elements modeling, simple analytical expressions for currents and current blockades (that do not include surface charges) are still good estimates for nanopores in this diameter range⁸. These simple equations are useful to rationalize the current scaling with different geometric parameters and to better understand how the coupled pore system behaves compared to the well-studied single pores. This is particularly helpful as a simple way to better rationalize the observed T_{1,2,3} and W_{1,2,3} levels in the [1,1] geometry.

Ionic conductance G through a single pore can be analytically estimated as:

$$G = \frac{I}{V} = {σ ( \frac{4 t}{π {d}^{2}} + \frac{1}{d} )}^{- 1} \qquad ( S7 )$$

, where d is the diameter of the nanopore, I is the open pore current without adding any analyte, V is the applied voltage and σ is the ionic solution conductivity (σ˜12 S/m for 1 M KCl at room temperature).

For the smaller-diameter and thinner 2D pore, this equation predicts G_{2D} shown in the color plot below (FIGS. 11A-11B), for a range of diameters d_{2D} and t_{2D} range from 0.7 to 2 nm. Assuming d_{2D}=2.3 nm-10 nm (suitable for dsDNA) and t_{2D}=1 nm, this simplified model gives G_{2D}˜25 nS-106 nS at 1 M KCl from Eq. (S7). For a SiN pore, G_{SiN} is shown in FIG. 11B for diameter range d_{SiN}=5 to 15 nm and thickness t_{SiN}=2 to 20 nm. For example, assuming d_{SiN}=15 nm and t_{SiN}=7 nm, this model gives G_{SiN}˜113 nS at 1 M KCl from Eq. (S7).

We can further estimate the conductance change, ΔG, using Eq. (S8):

$$Δ G = \frac{Δ I}{V} = σ \{ {( \frac{4 t}{π {d}^{2}} + \frac{1}{d} )}^{- 1} - {[ \frac{4 t}{π ( {d}^{2} - {d}_{DNA}^{2} )} + \frac{1}{\sqrt{{d}^{2} - {d}_{DNA}^{2}}} ]}^{- 1} \} \qquad ( S8 )$$

where d is the pore diameter, ΔI is the current blockade, V is the applied voltage and σ is the ionic solution conductivity.

For the smaller-diameter and thinner 2D pore this equation predicts ΔG shown in the color plot above (Equation S8). ΔG_{2D} is in the range 7.4-16.9 nS and ΔG_{SiN} is in the range 1.7-5.4 nS in these plots.

A rough model to capture some order of magnitude estimates includes two pores in series (this is a good model for uncoupled pores only). The open pore conductance is then:

$${G}_{two pores in series} = \frac{{G}_{SiN} {G}_{2 D}}{{G}_{2 D} + {G}_{SiN}} \qquad ( S9 )$$

The applied voltage is divided across the pores based on the ratio of their resistances. Therefore, a higher resistance pore experiences a larger voltage. The voltages V_{2D} and V_{SiN} across the two pores are:

$$\begin{matrix}{R}_{2 D} \\ {R}_{SiN}\end{matrix} \begin{matrix}V = {V}_{2 D} + {V}_{SiN} \\ {V}_{2 D} = V \frac{{R}_{2 D}}{{R}_{2 D} + {R}_{SiN}} \\ {V}_{SiN} = V \frac{{R}_{SiN}}{{R}_{2 D} + {R}_{SiN}}\end{matrix} \\ {V}_{2 D} = V \frac{{R}_{2 D}}{{R}_{2 D} + {R}_{SiN}} = V \frac{{G}_{SiN}}{{G}_{2 D} + {G}_{SiN}} , and {V}_{SiN} = V \frac{{G}_{2 D}}{{G}_{2 D} + {G}_{SiN}} \qquad ( S10 )$$

, where V is the applied voltage.

### Coupling Effects

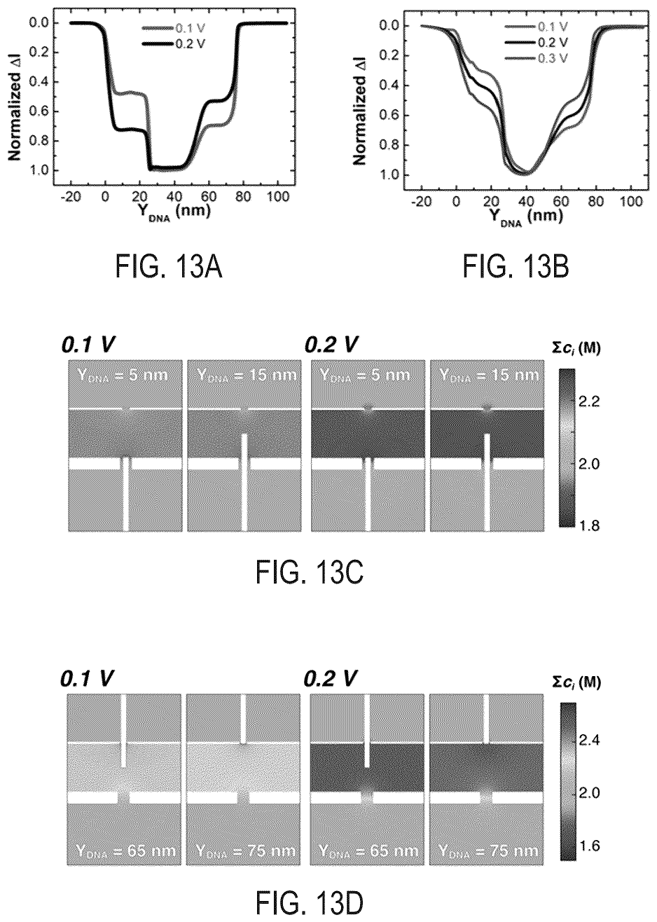

Providing a pair of two pores placed in proximity, allowing their respective electric fields to overlap, the relationship between sublevels, T₁ and T₃, gets more complicated. We have several possibilities: T₁˜T₃, T₁<T₃, and T₁>T₃. Briefly, the coupling is essential for small L. The asymmetric distribution of electric potential and ion concentration will affect the event depth in a non-trivial way. Since L=20 nm (FIG. 3) is small, and on the same order of magnitude as the pore diameters, the two-pore system can be also considered as one single asymmetric pore. One (2D layer) side has a smaller diameter, while the other (SiN layer) side has a larger diameter. FIGS. 13A, 13B present examples for tuning T₁ and T₃ levels to exhibit T₁˜T₃, T₁<T₃. and T₁>T₃. Counterintuitively, T₁>T₃ is possible even if the SiN pore is wider and thicker than the 2D pore. For example, if we simulate a smaller SiN pore at a higher voltage while fixing the geometry of the 2D pore, the T₁ level can be pushed deeper than T₃ (FIG. 13A). We attribute this to the concentration polarization effect during DNA translocation ^{9,10}. For example, the simulation result demonstrates the depletion of ions when DNA occupies the SiN pore, whereas ions accumulate when DNA occupies only the 2D pore. As shown in FIG. 13C, the depleted zone in the front of DNA becomes profound as voltages increase, resulting in a higher blockade in the SiN pore. On the other hand, when DNA only blocks the 2D pore (FIG. 13D), a significant accumulation of ions is observed in the trench region with higher voltages, hence a relatively shallower T₃ level. Overall, these combined effects lead to a deeper T₁ level and a shallower T₃ level. Similar reasoning leads to the other intermediate T-events and the W-events for shorter DNA. We note that the concentration-polarization-induced ion accumulation/depletion has been proposed previously in single nanopores ^{9,10,12} to elucidate the event shape. Once again, it is applied to the two-pore system for the first time. Other factors that could affect the signal magnitude and shape include pore geometries, surface charges of DNA and pore, and bulk concentration of the electrolyte solution. In contrast, for the decoupling case (pores far apart compared to their diameters), the system acts as two independent pores (FIG. 3A).

### Device Sensitivity to DNA Length and ΔG/ΔL_{DNA} for the [1,1] Configuration

This section discusses how signal patterns can be used to distinguish and size DNA molecules. In addition to the typical mean event depth and event duration, the characteristic patterns provide more sensitivity compared to single nanopore measurements. From G (at fixed Y_{DNA}=40 nm) vs. L_{DNA}, we estimate sensitivity ΔG/ΔL_{DNA}˜2-9 nS per kbp corresponding to FIG. 3C.

### Gel Electrophoresis on Measured DNA Samples

The testing result and analysis of gel electrophoresis are acquired from the Agilent 2200 TapeStation system (D5000 ScreenTape) for separating fragments up to 5 kbp, with reported sizing accuracy of ±10%¹⁴.

### Discussion: Other Factors That Affect Event Shapes

### Single Pore

We also simulate the blockade when a DNA molecule translocates through a single nanopore, as a control data set (FIGS. 17A-17B). A single 2D nanopore and a single SiN pore simulation data are presented. As expected, both blockades are clearly U-shaped. The percentage of current change obtained from a thinner single 2D pore is much larger than that from SiN.

### Hourglass Shape of the SiN Pore

It is well-documented that the SiN nanopores drilled in the TEM mode could have an hourglass-shape^{17,18}. Some of our GURU layers have SiN pores drilled in this mode (others are via RIE etching). To investigate the possible effect of SiN pore shape (cylinder or hourglass, both in a membrane of the same thickness) on observed signal shapes, we also model the SiN pore with an hourglass shape. The parameters include the effective thickness of 7 nm thickness, that correspond to one-third of the total SiN membrane thickness, determined by fabrication parameters. This hourglass shape was well documented previously and consistent with measurements. In our hourglass model, we assume the top part of the pore to be 2.5 times wider than the middle segment (i.e., 37.5 nm, see FIG. 18A)¹¹, thus defining an hourglass-like shape of the SiN nanopore. As shown in FIG. 18B, the shapes of current blockade are quite similar, indicating the hourglass shape of the SiN pore would not affect the qualitative behavior of translocation event.

### Surface Charge of DNA, Mixed Events (“Up” and “Down” Events)

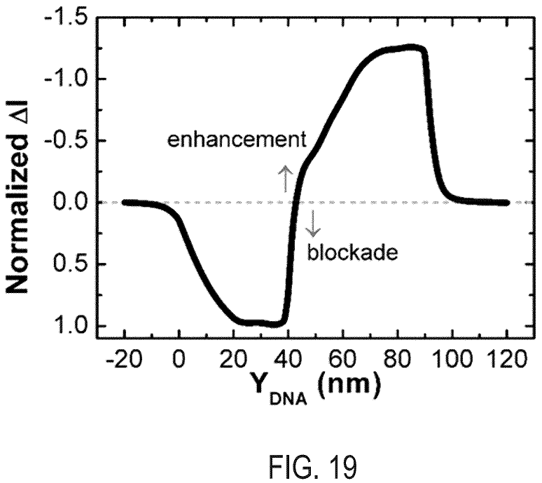

Beyond current blockade (“down”) events, a range of other event shapes such as up events and biphasic (up and down events) are possible and emerge from simulations, for different polymer charges and other relevant geometric and measurement parameters. For example, while DNA carries a bare charge density of −2e/bp, the screening of positive ions leads to a lower the effective charge density of DNA, which is typically 50%˜75% reduction of the bare charge, and this number can vary widely due to the environmental conditions. Assuming a charged rod having 2.2 nm in diameter, the bare surface charge density of DNA could be estimated as ˜−0.14 C/m². Compare to the factor of one-fourth adopted in the main text, we also consider a larger charge density for DNA (e.g., −0.1 C/m²) which means the bare charge is less screened. FIG. 19 shows a biphasic event that contains a blockade, followed by an enhancement when DNA starts to enter the 2D pore (Y_{DNA}>40 nm).

### Trajectory of DNA Translocation

Since the event shape may be affected by off-axis translocation of DNA, we also performed simple simulations considering the trajectory of DNA. For simplicity, the trajectory of DNA tested was chosen to follow the electric force lines. The electric force lines are obtained by solving the governing equations in the absence of DNA. In this case, the DNA orients itself before it is captured by the SiN pore. The obtained blockade still exhibited a T-shape and only showed a slight difference in magnitude (less than 2%) compared to the case assuming center axis translocation. However, we notice that the present model is simple and this question deserves further investigation with comprehensive modeling.

### Discussion: Feasibility of Stacking Ultrathin Multilayer GURU Devices

Beyond bilayers, here we estimate the open pore conductance scaling in multilayer coupled pores illustrated below. These could be in principle nanofabricated by stacking 2D material layers separated by thin spacers.

Not including any coupling effects, the total conductance from n layers with n ultrathin pores (one pore in each layer) with diameters and thicknesses dᵢ and tᵢ(i=1 to n) scales as:

$$\frac{1}{G} \sim {\sum }_{i = 1}^{n} \frac{1}{{G}_{i}} = {\sum }_{i = 1}^{n} \frac{1}{σ} ( \frac{4 {t}_{i}}{π {d}_{i}^{2}} + \frac{1}{{d}_{i}} ) \qquad ( S11 )$$

, where σ is the ionic solution conductivity in the pore. The experimental SNR and bandwidth will limit the maximum number of coupled pores that can be realistically integrated.

Atomically thin pores, low-noise chip platforms¹⁵ and higher salt concentration (e.g., 3 M KCl, σ˜30 S/m at room temperature¹⁶) will increase the signal and hence the possible number of pores, n. For example, assuming all pores are the same with dᵢ=3 nm-10 nm (suitable for dsDNA) and tᵢ=1 nm (i=1 to n), Gᵢ at 3 M KCl is approximately Gᵢ˜72 nS-280 nS and G˜7.2 nS-28 nS for n=10.

### Error in Pore Diameters

To calculate experimental pore diameters from AC-STEM images, we use ImageJ FFT bandpass filtering along with brightness thresholding to calculate pore area Aₚₒᵣₑ. Pore diameter d_{eff} is then calculated using d_{eff}=\sqrt{4Aₚₒᵣₑ/π}. Error is denoted by the mean difference of d_{eff} from the major and minor axes of the pore.

TABLE S2
Median dwell time of T-events for various chips recorded in 3M KCI.

| No. Chip | Voltage (mV) | SiN median (μs) | MoS₂ median (μs) |
|---|---|---|---|
| Chip N | 600 | 29.7 | 40.8 |
| | 700 | 37.1 | 42.6 |
| | 800 | 28.6 | 74.9 |
| Chip O | 600 | 18.1 | 29.0 |
| | 700 | 13.2 | 29.8 |
| | 800 | 11.5 | 29.7 |

### Discussion: GURU Device Statistics and Comments

After the ionic measurements, we inspected each device with optical microscopy to preliminarily confirm whether the 2D flakes and SiN membrane were still intact. About 30 devices are tested for DNA translocations and more were made to test separately on the feasibility of fabrication sequences. By design, most devices had desired 1-2-nm 2D pores on monolayer high-quality MoS₂ membranes. Various issues were observed over the series of measurements. First, unexpected high conductance. Some devices displayed larger open pore currents than expected. In a few chips, by accidentally exposing the 2D flake to the electron beam, bigger than desired, 2-3-nm in diameters, 2D pores were made. We also noticed an occasional fluid leak in the Elements fluid flow cell, where the gaskets would leak over the course of measurement if not placed in the desired orientations. Second, inconsistent wetting issue. About 13% of the devices were not able to wet. Some chips showed stable ionic current for a while, but then current overloaded the amplifier (either because of the fluid cell leak or the 2D flake broke or expanded in rare instances). Third, fabrication induced challenges and contamination. In one device, the transferred PMMA layer shifted on the chip surface during the drying process, resulting in partial misalignment of the MoS₂ flake with the thinned SiN region. Moreover, as shown in FIG. 2A, contaminations on the surface were also observed, deeming the device too dirty to fabricate nanopores.

TABLE 3

| type of material | d₁ (nm) ^{a} | d₂ (nm) ^{a} | L ^{b} | molecule |
|---|---|---|---|---|
| SiN/Si | 30 | 700 | ~2.3 μm | λ DNA, nanoparticles ^{c} |
| SiN/Si | 28 | 23 | 1.5 μm | dsDNA |
| SiN/SiN | ~10 | >230 | >2 μm | λ DNA |
| MoS₂/SiN | 2 | ~10 | 20 nm | dsDNA, ssDNA |

^{a} diameter,
^{b} interlayer separation.
^{c} For nanoparticle detection experiments, pore diameters were 50 nm (SiN) and 150 nm (Si).

### References

1. Kilic, M. S., Bazant, M. Z. & Ajdari, A. Steric effects in the dynamics of electrolytes at large applied voltages. II. Modified Poisson-Nernst-Planck equations. Phys. Rev. E 75, 021503 (2007).
2. Niedzwiecki, D. J. et al. Devices for Nanoscale Guiding of DNA through a 2D Nanopore. ACS Sens. 6, 2534-2545 (2021).
3. Hoogerheide, D. P., Garaj, S. & Golovchenko, J. A. Probing Surface Charge Fluctuations with Solid-State Nanopores. Phys. Rev. Lett. 102, 256804 (2009).
4. Ho, C. et al. Electrolytic transport through a synthetic nanometer-diameter pore. Proceedings of the National Academy of Sciences 102, 10445-10450 (2005).
5. Ai, Y., Liu, J., Zhang, B. & Qian, S. Field Effect Regulation of DNA Translocation through a Nanopore. Anal. Chem. 82, 8217-8225 (2010).
6. He, Y., Tsutsui, M., Fan, C., Taniguchi, M. & Kawai, T. Controlling DNA Translocation through Gate Modulation of Nanopore Wall Surface Charges. ACS Nano 5, 5509-5518 (2011).
7. Chou, Y.-C., Masih Das, P., Monos, D. S. & Drndić, M. Lifetime and Stability of Silicon Nitride Nanopores and Nanopore Arrays for Ionic Measurements. ACS Nano 14, 6715-6728 (2020).
8. Pérez, M. D. B. et al. Improved model of ionic transport in 2-D MoS ₂ membranes with sub-5 nm pores. Appl. Phys. Lett. 114, 023107 (2019).
9. Das, S., Dubsky, P., van den Berg, A. & Eijkel, J. C. T. Concentration Polarization in Translocation of DNA through Nanopores and Nanochannels. Phys. Rev. Lett. 108, 138101 (2012).
10. Carlsen, A. T., Zahid, O. K., Ruzicka, J., Taylor, E. W. & Hall, A. R. Interpreting the Conductance Blockades of DNA Translocations through Solid-State Nanopores. ACS Nano 8, 4754-4760 (2014).
11. Carson, S., Wilson, J., Aksimentiev, A. & Wanunu, M. Smooth DNA Transport through a Narrowed Pore Geometry. Biophysical Journal 107, 2381-2393 (2014).
12. Qiu, Y. et al. Highly Charged Particles Cause a Larger Current Blockage in Micropores Compared to Neutral Particles. ACS Nano 10, 8413-8422 (2016).
13. Chen, K. et al. Dynamics of driven polymer transport through a nanopore. Nat. Phys. 17, 1043-1049 (2021).
14. D5000 ScreenTape Assay for TapeStation Systems Quick Guide. (2018).
15. Balan, A. et al. Improving Signal-to-Noise Performance for DNA Translocation in Solid-State Nanopores at MHz Bandwidths. Nano Lett. 14, 7215-7220 (2014).
16. Danda, G. et al. Monolayer WS ₂ Nanopores for DNA Translocation with Light-Adjustable Sizes. ACS Nano 11, 1937-1945 (2017).
17. Kim, M. J., Wanunu, M., Bell, D. C. & Meller, A. Rapid Fabrication of Uniformly Sized Nanopores and Nanopore Arrays for Parallel DNA Analysis. Adv. Mater. 18, 3149-3153 (2006).
18. Rodríguez-Manzo, J. A., Puster, M., Nicolaï, A., Meunier, V. & Drndić, M. DNA Translocation in Nanometer Thick Silicon Nanopores. ACS Nano 9, 6555-6564 (2015).

### Aspects

The following Aspects are illustrative only and do not limit the scope of the present disclosure or the appended claims. Any part or parts of any one or more Aspects can be combined with any part or parts of any one or more other Aspects.

Aspect 1. A molecular analysis component, comprising: a first substrate having a first nanopore extending therethrough and the first nanopore having a diameter; and a second substrate having a second nanopore extending therethrough and the second nanopore having a diameter, the first and second nanopores both extending in a direction, and the first and second nanopores are separated by a distance of from about 0.5 nm to about 500 nm as measured along the direction, the distance optionally being of from about 5 to about 250 nm.

The distance (measured along the direction) between the first nanopore and the second nanopore can be, e.g., from about 0.5 nm to about 500 nm, from about 1 nm to about 400 nm, from about 3 nm to about 350 nm, from about 5 nm to about 250 nm or 300 nm, from about 8 nm to about 250 nm, from about 10 nm to about 200 nm, from about 10 nm to about 200 nm, from about 15 nm to about 150 nm, and all intermediate values and subranges.

Aspect 2. The molecular analysis component of Aspect 1, wherein a line drawn along the direction passes through the first nanopore and the second nanopore. In some cases, the center of the first nanopore is in line (as measured along the direction) with the center of the second nanopore, e.g., as shown in FIG. 4A. In some case, the center of the first nanopore is within about 5 nm (as measured along the direction) of the center of the second nanopore. In some cases, the first nanopore and the second pore are in at least partial register with one another; i.e., a portion of one of the nanopores overlies a portion of the other.

Aspect 3. The molecular analysis component of Aspect 1, wherein a line drawn along the direction passes through only one of the first nanopore and the second nanopore. This offset configuration is shown in, e.g., FIG. 1A.

Aspect 4. The molecular analysis component of any one of Aspects 1 to 3, wherein at least one of the first substrate and the second substrate comprises a plurality of nanopores extending therethrough. In some cases, the first substrate comprises a single nanopore, and the second substrate comprises a plurality of nanopores. In some cases, the first substrate comprises a plurality of nanopores, and the second substrate comprises a single nanopore. In some cases, the first substrate and the second substrate each comprises a plurality of nanopores.

Aspect 5. The molecular analysis component of Aspect 4, wherein the first substrate and the second substrate comprise a different number of nanopores.

Aspect 6. The molecular analysis component of any one of Aspects 1 to 5, wherein the diameter of the first nanopore is from about 0.3 nm to about 100 nm. The diameter of the first nanopore can be, e.g., from about 0.3 nm to about 100 nm, from about 0.5 nm to about 75 nm, from about 1 nm to about 50 nm, from about 1 nm to about 40 nm, from about 1.3 nm to about 35 nm, from about 1.7 nm to about 30 nm, from about 2 nm to about 25 nm, from about 2.3 nm to about 20 nm, from about 2.7 nm to about 18 nm, or even from about 3 nm to about 13 nm, and all intermediate values and subranges. Without being bound to any particular theory or embodiment, the second nanopore can be, e.g., from about 0.3 nm to about 100 nm, from about 0.5 nm to about 75 nm, from about 1 nm to about 50 nm, from about 1 nm to about 40 nm, from about 1.3 nm to about 35 nm, from about 1.7 nm to about 30 nm, from about 2 nm to about 25 nm, from about 2.3 nm to about 20 nm, from about 2.7 nm to about 18 nm, or even from about 3 nm to about 13 nm, and all intermediate values and subranges.

Without being bound to any particular theory or embodiment, one can utilize a comparatively small pore size in the bottom layer of the disclosed devices, as smaller pores can in some instances facilitate both guiding and linearizing molecules. This can in turn enhance the resolution in distinguishing DNA lengths. As an example, one may utilize pores in the bottom layer having a diameter of less than 10 nm. Pores in the top layer can vary in diameter; in some instances, one may utilize pore diameters of from 1.5-2 nm for ssDNA applications and utilize pore diameters of 2-3 nm for dsDNA applications; such sizes can match molecule sizes. In some embodiments, the ratio of the diameter of the pores in the upper layer to the diameter of the pores in the lower layer can be from about 0.1 to about 0.4.

Again without being bound to any particular theory or embodiment, when a pore is smaller in diameter, DNA can tend to avoid folding, allowing the DNA to stretch like a rigid rod before threading into the sensing pore. Further, long DNA molecules can exist in a coiled conformation/polymer blob (which can be hundreds of nanometers in size), which is larger than the nanometer-sized sensing pore. Therefore, smaller pores in the bottom layer are also beneficial for disentangling the blob before the DNA end is pulled through the sensing pore.

One can also vary the number of pores and spacing in the bottom layer. Without being bound to any particular theory, a higher number of pores can enhance capture rates and improves the sensitivity of the device. Also without being bound to any particular theory, a wider pore spacing can prevent double-threading through adjacent pores in the bottom layer, thereby minimizing clogging.

As an example, non-limiting embodiment, one can fabricate 20-25 pores in the bottom layer, with the pores being separated from one another by 100 nm or 200 nm. This results in density of 1 or 2 pores per 10000 nm²; pores can be present at, for example, from 1 to 5 pores per 10000 nm², although this is not a requirement. Without being bound to any particular theory or embodiment, one can utilize pore spacing of at least 100 nm or 200 nm; pores can be spaced apart by 100, 150, 200, 250, 300, 350, 400, 450, 500, 600, 700, 800, 900, or even by 1000 nm. Pores can be spaced by 1000 nm, 1250 nm, 1500 nm, 1750 nm, 2000 nm, 2250 nm, 2500 nm, 2750 nm, or even 3000 nm. Pores

Aspect 7. The molecular analysis component of any one of Aspects 1 to 6, wherein the diameter of the first nanopore differs from the diameter of the second nanopore. As an example, the first nanopore can have a larger diameter than the second nanopore. As an example, the second nanopore can have a larger diameter than the first nanopore. As a further example, the first nanopore and the second nanopore can have the same diameter.

Aspect 8. The molecular analysis component of any one of Aspects 1 to 9, wherein (i) the first substrate comprises a thick region having a recess defined therein, (ii) the first substrate comprises a thinned region through which the first nanopore extends, (iii) the first nanopore is in fluid communication with the recess of the first substrate, and (iv) the recess defines a width greater than the diameter of the first nanopore.

An example such component is provided in FIGS. 26A-26B. As shown, a component can include a first substrate (e.g., SiN) that includes a thick region having a recess defined therein. A first nanopore extends through the thinned region of the first substrate, with the first nanopore having a diameter that is less than the width of the recess; the diameter and width can be measured perpendicular to the direction in which the first nanopore extends. As shown, the second substrate has a second nanopore extending therethrough. (It should be understood that the materials and electrical diagram on FIGS. 26A-26B are illustrative only and are not limiting.)

Aspect 9. The molecular analysis component of Aspect 8, wherein the second substrate is superposed over the first substrate such that the second nanopore is in register with the recess of the first substrate.

Aspect 10. The molecular analysis component of any one of Aspects 8 to 9, wherein the thick region of the first substrate has a thickness of from about 0.5 nm to about 500 nm. The thickness can be, e.g., from about 0.5 nm to about 500 nm, from about 1 nm to about 400 nm, from about 5 nm to about 350 nm, from about 8 nm to about 300 nm, from about 10 nm to about 275 nm, from about 20 nm to about 200 nm, from about 35 nm to about 300 nm, or even from about 50 nm to about 250 nm, and all intermediate values and subranges.

Aspect 11. The molecular analysis component of any one of Aspects 10 to 12, wherein the thinned region of the first substrate has a thickness of from about 0.5 nm to about 200 nm. The thickness can be, e.g., from about 0.5 nm to about 200 nm, from about 1 nm to about 150 nm, from about 3 nm to about 125 nm, from about 5 nm to about 100 nm, from about 8 nm to about 75 nm, from about 10 nm to about 50 nm, or even from about 20 nm to about 45 nm, and all intermediate values and subranges.

Aspect 12. The molecular analysis component of any one of Aspects 1 to 11, wherein at least one of the first substrate and the second substrate comprises a monolayer material. It should be understood that either of the first or second substrate can comprise a monolayer material; in some cases, both the first substrate and the second substrate comprises a monolayer material. The first substrate and the second substrate can comprise the same monolayer material; alternatively, the first substrate and the second substrate can comprise different monolayer materials. Example monolayer materials include, e.g., MoS₂, WS₂, boron nitride (which can be hexagonal boron nitride), a MXene, graphene, and the like. Without being bound to any particular theory or embodiment, a comparatively hydrophilic material such as hexagonal boron nitride can give rise to more effective pore wetting, which can in turn improve data collection. Exfoliation and PDMS transfer methods can also reduce contamination.

Without being bound to any particular theory or embodiment, it can be useful to wet the nanopores when measuring ionic current. For traditional SiN pores, one can clean the membrane using a piranha solution. One can also utilize, for example, MoS₂, WS₂, and hBN. The hydrophilicity of a hBN membrane can be significantly enhanced through UV-ozone treatment, due to its oxidation resistance compared to other 2D materials such as graphene and MoS₂. MXene materials can also be intrinsically hydrophilic.

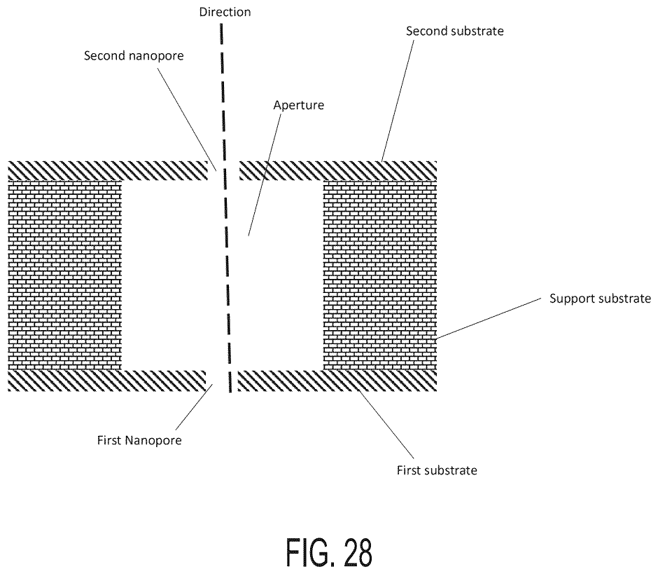

FIG. 28 provides an example component. As shown, a component can include a monolayer first substrate and a monolayer second substrate, with a support substrate therebetween. The support substrate can have an aperture extending therethrough. As shown, the support substrate can be constructed so as to place the first nanopore into fluid communication with the second nanopore. The fluid communication can be across an opening formed in a substrate between the first substrate and the second substrate, e.g., as shown by the aperture formed in the support substrate of FIG. 28.

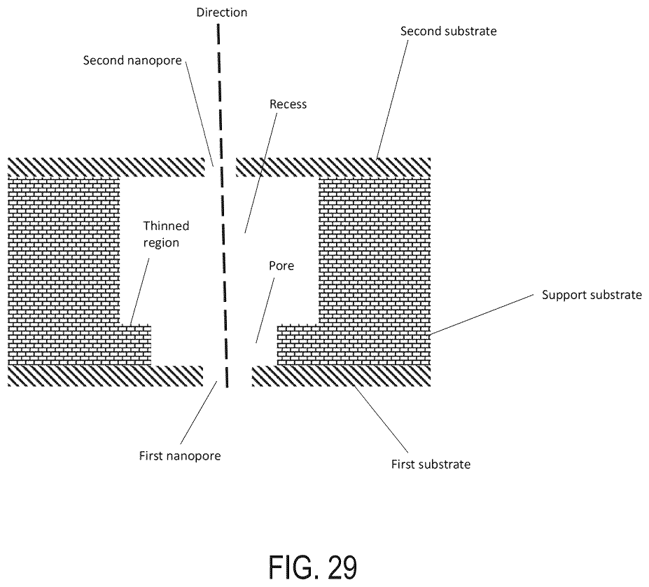

A further example component is shown in FIG. 29. As shown in that figure, a component can include a monolayer first substrate and a monolayer second substrate, with a support substrate therebetween. The support substrate can have a recess formed therein, with a pore extending into the recess. As shown, the support substrate can be constructed so as to place the first nanopore into fluid communication with the second nanopore; the first nanopore and the second nanopore can be in register with one another, although this is not a requirement. The first and second substrates can be the same monolayer material, but can also be formed of different monolayer materials. As shown, a pore can be formed in the recess of the support substrate, with the pore being formed through the thinned region and the pore being in register with the first nanopore of the first substrate.

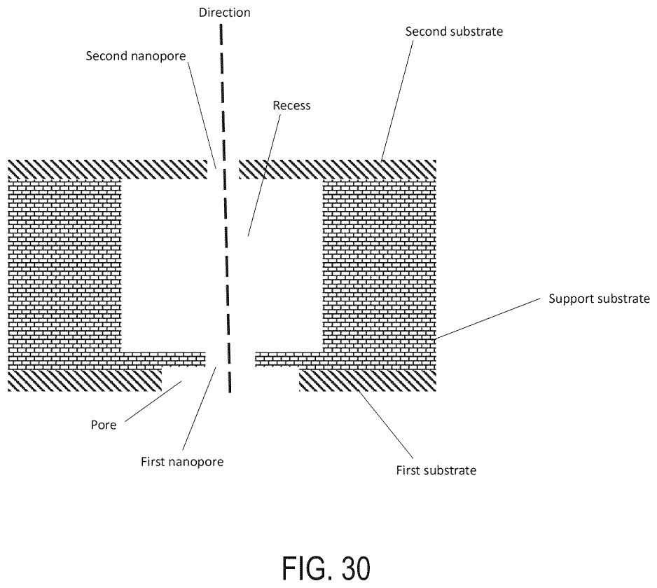

A further example component is shown in FIG. 30. As shown in that figure, a component can include a monolayer first substrate and a monolayer second substrate, with a support substrate therebetween. The support substrate can have a recess formed therein, with a pore extending into the recess. As shown, the support substrate can be constructed so as to place the first nanopore into fluid communication with the second nanopore; the first nanopore and the second nanopore can be in register with one another, although this is not a requirement. The first and second substrates can be the same monolayer material, but can also be formed of different monolayer materials.

As shown in FIG. 30, the first nanopore can be formed in the support substrate, with a comparatively wider pore being formed in the first substrate, the comparatively wider pore being in register with the first nanopore.

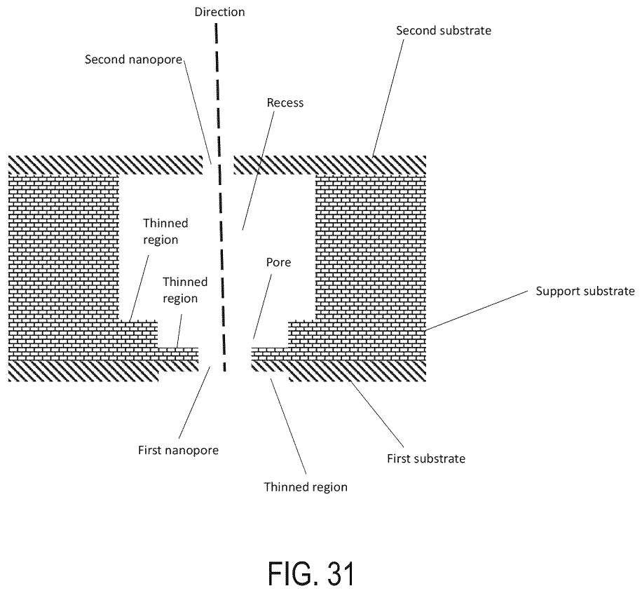

A further example component is shown in FIG. 31. As shown in that figure, a component can include a monolayer first substrate and a monolayer second substrate, with a support substrate therebetween. The support substrate can have a recess formed therein, with a pore extending into the recess. As shown, the support substrate can be constructed so as to place the first nanopore into fluid communication with the second nanopore; the first nanopore and the second nanopore can be in register with one another, although this is not a requirement. The first and second substrates can be the same monolayer material, but can also be formed of different monolayer materials.

As shown in FIG. 31, the support substrate can include stepped or thinned regions, with a nanopore being formed through the thinnest of the thinned regions. The first substrate can also include a thinned region, with a nanopore being formed in the thinned region. Without being bound to any particular theory, the first nanopore can be formed by drilling through the thinned regions of the first substrate and the support substrate.

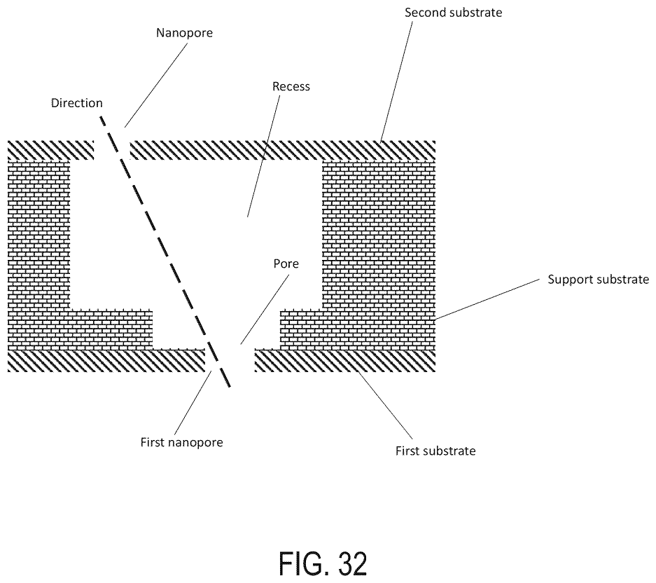

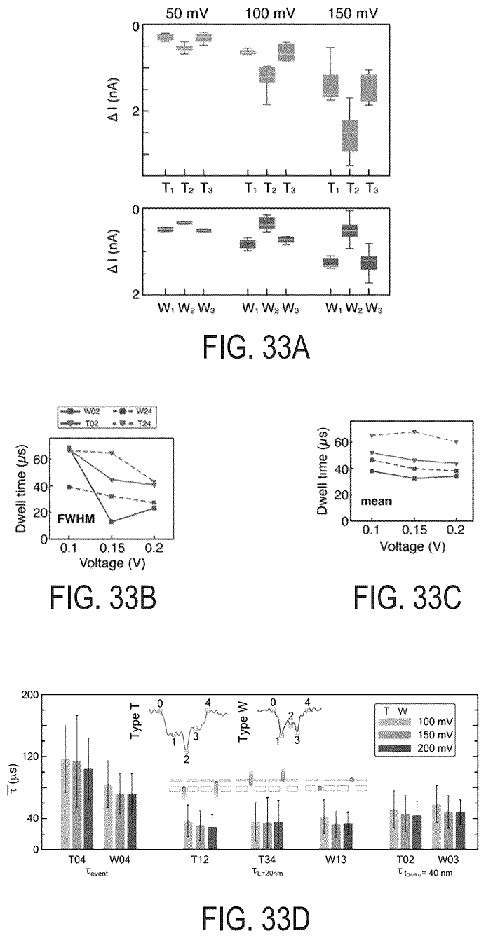

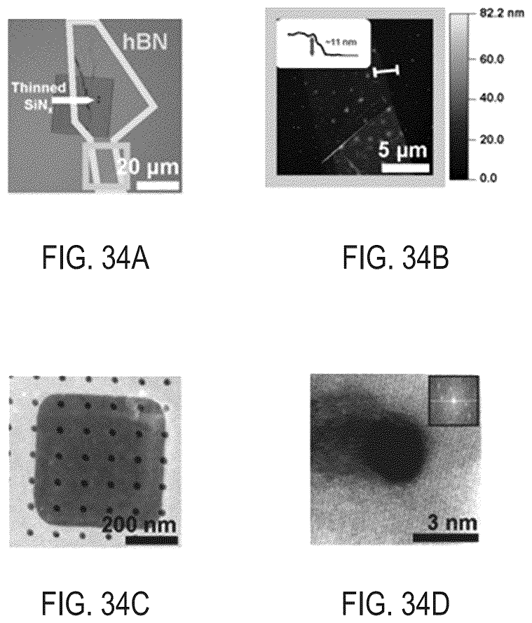

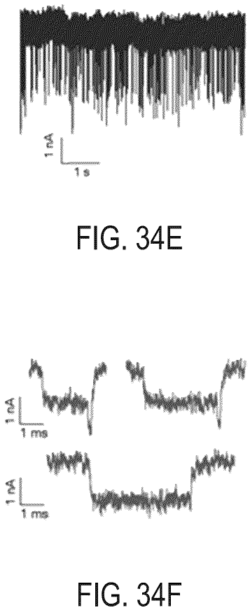

An additional example embodiment is provided in FIG. 32. As shown in that figure, a component can include a monolayer first substrate and a monolayer second substrate, with a support substrate therebetween. The support substrate can have a recess formed therein, with a pore extending into the recess. As shown, the support substrate can be constructed so as to place the first nanopore into fluid communication with the second nanopore; the first nanopore and the second nanopore can be offset from one another, although this is not a requirement. The first and second substrates can be the same monolayer material, but can also be formed of different monolayers material.

Aspect 13. The molecular analysis component of any one of Aspects 1 to 12, wherein at least one of the first substrate and the second substrate comprises at least one of silicon oxide, silicon nitride, aluminum oxide, or hafnium oxide.

Aspect 14. A method, comprising translocating a molecule through the molecular analysis component of any one of Aspects 1 to 13.

Aspect 15. The method of Aspect 14, further comprising collecting at least one signal related to the translocation of the molecule and relating the at least one signal to a structural feature of the molecule. The signal can be, e.g., a value of or a change in value of a current, a voltage, a capacitance, and the like. The signal can be correlated to a feature of the molecule, e.g., the presence or absence of a particular monomer unit. As but one example, the signal can be correlated to the presence or absence of a nucleic acid and/or an amino acid. The signal profile evolved from the translocation of the molecule can be related to the structure of at least a portion of the molecule.

Aspect 16. A method, comprising: translocating a molecule through (i) a first nanopore extending through a first substrate and (ii) a second nanopore extending through a second substrate, the first and second nanopores both extending along a direction, the first and second nanopores being separated by a distance as measured along the direction; and collecting at least one signal related to the translocation of the molecule through at least one of the first nanopore and the second nanopore, the molecule optionally comprising a polynucleotide.

Suitable first substrates, second substrates, first nanopores, second nanopores, and distances are described elsewhere herein.

Aspect. 17. The method of Aspect 16, wherein the distance is less than a persistence length of the molecule.

Aspect 18. The method of any one of Aspects 16 to 17, wherein a line drawn along the direction passes through the first nanopore and the second nanopore.

Aspect 19. The method of any one of Aspects 16 to 17, wherein a line drawn along the direction passes through only one of the first nanopore and the second nanopore.

Aspect 20. The method of any one of Aspects 16 to 19, wherein (i) the first substrate comprises a thick region having a recess defined therein, (ii) the first substrate comprises a thinned region through which the first nanopore extends, (iii) the first nanopore is in fluid communication with the recess of the first substrate, and (iv) the first diameter defines a diameter and the recess defines a width greater than the diameter of the first nanopore.

Aspect 21. The method of Aspect 20, wherein the second substrate is superposed over the first substrate such that the second nanopore is in register with the recess of the first substrate.

Aspect 22. The method of any one of Aspects 16 to 21, wherein the first nanopore defines a diameter greater than a diameter of the second nanopore.

Aspect 23. The method of any one of Aspects 16 to 22, wherein the second substrate is a monolayer material.

Aspect 24. The method of any one of Aspects 16 to 23, further comprising relating the at least one signal to a structural feature of the molecule.

Aspect 25. The method of any one of Aspects 16 to 24, wherein the at least one signal comprises a signal related to translocation of the molecule through the first nanopore and a signal related to translocation of the molecule through the second nanopore.

It should be understood that a component according to the present disclosure can include nanopores formed in more than two substrates. As shown in FIG. 25, a component can include n substrates (e.g., wherein n ranges from 1 to i), in which each substrate comprises one or more nanopores, and each substrate is spaced by a distance from the adjacent substrate or substrates. In this fashion, one can construct tiered devices in which a given molecule is translocated through a plurality of nanopores arranged in n tiered substrates, arranged one above the other. Without being bound to any particular theory, n can be any of 2, 3, 4, 5, 6, 7, 8, 9, or 10.

What is claimed:

1. A molecular analysis component, comprising:
a first substrate having a first nanopore extending therethrough and the first nanopore having a diameter; and
a second substrate having a second nanopore extending therethrough and the second nanopore having a diameter,
the first and second nanopores both extending in a direction, and
the first and second nanopores are separated by a distance of from about 0.5 nm to about 500 nm as measured along the direction, the distance optionally being of from about 5 to about 250 nm.

2. The molecular analysis component of claim 1, wherein a line drawn along the direction passes through the first nanopore and the second nanopore.

3. The molecular analysis component of claim 1, wherein a line drawn along the direction passes through only one of the first nanopore and the second nanopore.

4. The molecular analysis component of claim 1, wherein at least one of the first substrate and the second substrate comprises a plurality of nanopores extending therethrough.

5. The molecular analysis component of claim 4, wherein the first substrate and the second substrate comprise a different number of nanopores.

6. The molecular analysis component of claim 1, wherein the diameter of the first nanopore is from about 0.3 nm to about 100 nm.

7. The molecular analysis component of claim 1, wherein the diameter of the first nanopore differs from the diameter of the second nanopore.

8. The molecular analysis component of any claim 1, wherein (i) the first substrate comprises a thick region having a recess defined therein, (ii) the first substrate comprises a thinned region through which the first nanopore extends, (iii) the first nanopore is in fluid communication with the recess of the first substrate, and (iv) the recess defines a width greater than the diameter of the first nanopore.

9. The molecular analysis component of claim 8, wherein the second substrate is superposed over the first substrate such that the second nanopore is in register with the recess of the first substrate.

10. The molecular analysis component of claim 8, wherein the thick region of the first substrate has a thickness of from about 0.5 nm to about 500 nm.

11. The molecular analysis component of claim 10, wherein the thinned region of the first substrate has a thickness of from about 0.5 nm to about 200 nm.

12. The molecular analysis component of claim 1, wherein at least one of the first substrate and the second substrate comprises a monolayer material.

13. The molecular analysis component of claim 1, wherein at least one of the first substrate and the second substrate comprises at least one of silicon oxide, silicon nitride, aluminum oxide, or hafnium oxide.

14. A method, comprising translocating a molecule through the molecular analysis component of claim 1.

15. The method of claim 14, further comprising collecting at least one signal related to the translocation of the molecule and relating the at least one signal to a structural feature of the molecule.

16. A method, comprising:
translocating a molecule through (i) a first nanopore extending through a first substrate and (ii) a second nanopore extending through a second substrate,
the first and second nanopores both extending along a direction,
the first and second nanopores being separated by a distance as measured along the direction; and
collecting at least one signal related to the translocation of the molecule through at least one of the first nanopore and the second nanopore,
the molecule optionally comprising a polynucleotide.

17. The method of claim 16, wherein the distance is less than a persistence length of the molecule.

18. The method of claim 16, wherein a line drawn along the direction passes through the first nanopore and the second nanopore.

19. The method of claim 16, wherein a line drawn along the direction passes through only one of the first nanopore and the second nanopore.

20. The method of claim 16, wherein (i) the first substrate comprises a thick region having a recess defined therein, (ii) the first substrate comprises a thinned region through which the first nanopore extends, (iii) the first nanopore is in fluid communication with the recess of the first substrate, and (iv) the recess defines a width greater than a diameter of the first nanopore.

21. The method of claim 20, wherein the second substrate is superposed over the first substrate such that the second nanopore is in register with the recess of the first substrate.

22. The method of claim 16, wherein the first nanopore defines a diameter greater than a diameter of the second nanopore.

23. The method of claim 16, wherein the second substrate is a monolayer material.

24. The method of claim 16, further comprising relating the at least one signal to a structural feature of the molecule.

25. The method of claim 16, wherein the at least one signal comprises a signal related to translocation of the molecule through the first nanopore and a signal related to translocation of the molecule through the second nanopore.
